(19) United States
(12) Patent Application Publication
(10) Pub. No.: US 2014/0142095 A1
(43) Pub. Date: May 22, 2014

(54) AGENTS USEFUL FOR TREATING FRIEDREICH'S ATAXIA AND OTHER NEURODEGENERATIVE DISEASES

(71) Applicants: Gino Cortopassi, Davis, CA (US); Sunil Sahdeo, Davis, CA (US)

(72) Inventors: Gino Cortopassi, Davis, CA (US); Sunil Sahdeo, Davis, CA (US)

(73) Assignee: SERKET PHARMA, LLC, Davis, CA (US)

(21) Appl. No.: 14/114,183

(22) PCT Filed: Apr. 27, 2012
(86) PCT No.: PCT/US12/35668

§ 371 Date: Jan. 15, 2014

Related U.S. Application Data
(60) Provisional application No. 61/480,170, filed on Apr. 28, 2011.

(51) Int. Cl. A61K 31/4453; A61K 31/225; A61K 31/345; A61K 31/5415

(52) U.S. Cl. CPC A61K 31/4453; A61K 31/5415; A61K 31/225; A61K 31/345
USPC 514/224.8; 514/317; 514/547

(57) ABSTRACT

This invention provides methods of identifying agents useful to prevent, ameliorate or treat one or more symptoms of Friedreich's ataxia or other neurodegenerative disease, and methods of employing the identified agents to prevent, reduce, delay or inhibit one or more systems of Friedreich's ataxia or other neurodegenerative disease.

### CROSS-REFERENCE TO RELATED APPLICATION

This application is a nonprovisional and claims the benefit of 61/480,170, filed Apr. 28, 2011, which is incorporated by reference in its entirety for all purposes.

### FIELD

The present invention relates to the discovery and use of compounds to prevent, reduce, delay or inhibit one or more symptoms of Friedreich's ataxia or other neurodegenerative disease.

### BACKGROUND

Friedreich's ataxia (FRDA) is the most common autosomal recessive inherited movement disorder, with six thousand Americans (and many more thousands worldwide) diagnosed with this disease. The clinical manifestations of FRDA are the result of deficiency of the frataxin protein. The phenotype of FRDA includes degeneration and demyelination of the spinocerebellar dorsal root ganglion neurons, and most Friedreich's patients are wheelchair-bound by age 20 (Dun and Brice, Curr Opin Neurol (2000) 13:407-413). A progressive, usually lethal cardiomyopathy also occurs (Albano, et al., Arq Bras Cardiol (2002) 78:444-451). FRDA phenocopies the glutathione transporter disease and Vitamin E transporter disease, supporting the idea that all three are diseases of oxidative stress (Benomar, et al., J Neurol Sci (2002) 198:25-29). About 25 percent of people with Friedreich's ataxia have an atypical form that begins after age 25. Affected individuals who develop Friedreich ataxia between ages 26 and 39 are considered to have late-onset Friedreich ataxia (LOFA). When the signs and symptoms begin after age 40 the condition is called very late-onset Friedreich ataxia (VLOFA). LOFA and VLOFA usually progress more slowly than typical Friedreich ataxia.

There is currently no approved therapy for Friedreich's ataxia.

### SUMMARY OF THE CLAIMED INVENTION

The invention provides a method for reducing, delaying or inhibiting Friedreich's ataxia or other neurodegenerative disease in a subject in need thereof comprising administering to the subject an effective amount of a compound conforming to formula (II) as described herein or a pharmaceutically acceptable salt thereof. Optionally, the compound of formula (II) is a compound conforming to formula (IIA) or a pharmaceutically acceptable salt thereof. Optionally, the compound is of formula (II) a compound conforming to formula (IIB) or a pharmaceutically acceptable salt thereof. Optionally, the compound of formula (II) is dyclonine or a pharmaceutically acceptable salt thereof.

Optionally, the dyclonine is administered in a dose of 1-500 mg/subject, preferably at least 100 mg/subject. Optionally, the dyclonine is administered in a dose of a least 1 mg/kg. Optionally, the dyclonine is formulated as a controlled-release composition. Optionally, the dyclonine is administered intramuscularly, intravenously, subcutaneously or orally. Optionally, the dyclonine is in the form of a pharmaceutically acceptable salt other than HCl.

Optionally, the subject is co-administered an effective amount of DMF or methylene blue or a pharmaceutically acceptable salt thereof. Optionally, the subject is free of other known diseases amenable to treatment with dyclonine. Optionally, the subject is monitored for an increase in level of frataxin responsive to the administering.

The invention further provides a controlled-release formulation of dyclonine.

The invention further provides a single-use formulation of dyclonine containing at least 100 mg dyclonine.

The invention further provides a method for reducing, delaying or inhibiting Friedreich's ataxia or other neurodegenerative disease in a subject in need thereof comprising administering to the subject an effective amount of a compound of formula (I) as further defined herein or a pharmaceutically acceptable salt thereof. Optionally, the compound of formula (I) is dimethylfumarate.

The invention further provides a method for reducing, delaying or inhibiting Friedreich's ataxia or other neurodegenerative disease in a subject in need thereof comprising administering to the subject an effective amount of leuco-methylene blue and acetyl-methylene blue, 2-chlorophenothiazine, phenothiazine, toluidine blue, tolonium chloride, toluidine blue O, seleno toluidine blue, methylene green, chlorpromazine, sulphoxide chlorpromazine, sulphone chlorpromazine, chlordiethazine promethazine, thioproperazine, prochlorperazine, pipotiazine, dimetotiazine, propericiazine, metazionic acid, oxomemazine neutral red, iminostilbene, and imipramine or a compound of formula (III), or a compound of formula (IV) as further described herein, or a pharmaceutically acceptable salt of either of these. Optionally, the compound of formula (III) is methylene blue or a pharmaceutically acceptable salt thereof. Optionally, the subject is monitored for an increase in level of frataxin responsive to the administering.

The invention further provides a method of screening agents for activity useful in treating Friedreich's ataxia. The method comprises (a) determining whether agents agonize the thioredoxin reductase and NRF2 pathway and, (b) if an agent does agonize the NRF2 pathway, determining whether the agent is effective in a cellular or animal model of Friedreich's ataxia. Optionally, the determining in step (b) comprises determining whether the agent increases a level of frataxin. Optionally, step (b) is performed in a mouse encoding a mutated human frataxin and having a knocked out endogenous frataxin gene.

The invention further provides a method for reducing, delaying or inhibiting Friedreich's ataxia in a subject in need thereof. The method comprises administering to the subject an effective amount of an agonist of the NRF2 pathway. The agonist can cross the blood brain barrier.

The invention further provides a method for reducing, delaying or inhibiting a neurodegenerative disease, heart or lung disease. The method comprises administering to a subject in need thereof an agent that agonizes the NRF2 pathway and thereby reducing, delaying or inhibiting the neurodegenerative disease. The agent is a compound conforming to formula I, II, IIA, IIB, III, or (IV) or a pharmaceutically acceptable salt thereof. The neurodegenerative disease can be an neurodegenerative disease that results from protein aggregates, and agonizing of the NRF2 pathway inhibits an inflammatory response to amyloid deposits. For example, the disease is Alzheimer's. Optionally, the subject is monitored for an increased level of frataxin responsive to the administering.

The invention further provides methods for preventing, reducing, delaying or inhibiting Friedreich's ataxia or other neurodegenerative disease in a subject in need thereof. In some embodiments, the methods comprise administering to the subject an effective amount of an agent selected from the group consisting of anethole, aspartame, cephradine, cotinine, dexamethasone, dimethyl fumarate, diphenhydramine, dyclonine, ebselen, isoflupredone, meclocycline, mepartricin, methylene blue, nifursol, oxfendazole, sulfisoxazole, thioctic acid, tolonium cl, tryptophan/3-hydroxyanthrandate, and yohimbine.

The invention further provides methods for preventing, reducing, delaying or inhibiting one or more symptoms of Friedreich's ataxia or other neurodegenerative disease in a subject in need thereof. In some embodiments, the methods comprise administering to the subject an effective amount of an agent selected from the group consisting of anethole, aspartame, cephradine, cotinine, dexamethasone, dimethyl fumarate, diphenhydramine, dyclonine, ebselen, isoflupredone, meclocycline, mepartricin, methylene blue, nifursol, oxfendazole, sulfisoxazole, thioctic acid, tolonium cl, tryptophan/3-hydroxyanthranilate, and yohimbine

In some embodiments, the disease is Friedreich's ataxia and symptoms are selected from the group consisting of muscle weakness in the arms and legs, loss of coordination, loss of deep tendon reflexes, loss of extensor plantar responses, loss of vibratory and proprioceptive sensation, vision impairment, involuntary and/or rapid eye movements, hearing impairment, slurred speech, curvature of the spine (scoliosis), high plantar arches (pes cavus deformity of the foot), carbohydrate intolerance, diabetes mellitus, and heart disorders (e.g., atrial fibrillation, tachycardia (fast heart rate), hypertrophic cardiomyopathy, cardiomegaly, symmetrical hypertrophy, heart murmurs, and heart conduction defects).

In some embodiments, the agent is an inhibitor of the arachidonic acid pathway. For example, in various embodiments, the agent is selected from the group consisting of dexamethasone and diphenhydramine and mixtures and analogs thereof.

In some embodiments, the agent is a sulfur-containing compound affecting mitochondria. For example, in various embodiments, the agent is selected from the group consisting of thioctic acid, lipoic acid and lipoamide and mixtures and analogs thereof.

In some embodiments, the agent is an antioxidant. For example, in various embodiments, the agent is ebselen, or an analog thereof.

In some embodiments, the agent is an inducer of the Nrf2 antioxidant response pathway, that is neuroprotective. For example, in various embodiments, the agent is selected from the group consisting of anethole, aspartame, dexamethasone, dimethyl fumarate, dyclonine, ebselen, mepartricin, methylene blue, nifursol, oxfendazole, sulfisoxazole, thioctic acid, tolonium cl, tryptophan/3-hydroxyanthranilate, yohimbine.

In some embodiments, the agent promotes or induces the mitochondrial ferredoxin/adrenodoxin pathway. For example, in various embodiments, the agent is selected from the group consisting of isoflupredone, and mixtures and analogs thereof.

In some embodiments, the agent increases expression levels of frataxin. For example, the agent can be any of anethole, aspartame, cephradine, cotinine, dexamethasone, dimethyl fumarate, diphenhydramine, dyclonine, ebselen, isoflupredone, meclocycline, mepartricin, methylene blue, nifursol, oxfendazole, sulfisoxazole, thioctic acid, tolonium cl, tryptophan/3-hydroxyanthranilate, yohimbine and mixtures and analogs thereof.

In some embodiments, the agent inhibits the activity of thioredoxin reductase, to which cells respond by increasing Nrf2 transcription factor, which induces a neuroprotective response, including the induction of frataxin. For example, the agent can be any of anethole, aspartame, cephradine, cotinine, dexamethasone, dimethyl fumarate, diphenhydramine, dyclonine, ebselen, isoflupredone, meclocycline, mepartricin, methylene blue, nifursol, oxfendazole, sulfisoxazole, thioctic acid, tolonium cl, tryptophan/3-hydroxyanthranilate, yohimbine and mixtures and analogs thereof.

In some embodiments, the agent increases mitochondrial iron-sulfur cluster biogenesis. For example, the agent can be any of anethole, aspartame, cephradine, cotinine, dexamethasone, dimethyl fumarate, diphenhydramine, dyclonine, ebselen, isoflupredone, meclocycline, mepartricin, methylene blue, nifursol, oxfendazole, sulfisoxazole, thioctic acid, tolonium cl, tryptophan/3-hydroxyanthranilate, yohimbine and mixtures and analogs thereof.

In some embodiments, the agent inhibits Histone Lysine Methyltransferase activity, which increases the expression of multiple neuroprotective genes including frataxin. For example, the agent can be any of anethole, aspartame, cephradine, cotinine, dexamethasone, dimethyl fumarate, diphenhydramine, dyclonine, ebselen, isoflupredone, meclocycline, mepartricin, methylene blue, nifursol, oxfendazole, sulfisoxazole, thioctic acid, tolonium cl, tryptophan/3-hydroxyanthranilate, yohimbine and mixtures and analogs thereof.

In some embodiments, the subject is a human.

In some embodiments, the subject is exhibiting symptoms of Friedreich's ataxia. In some embodiments, the subject is asymptomatic. In some embodiments, the subject has been diagnosed with Friedreich's ataxia.

In some embodiments, the agent is administered systemically. In some embodiments, the agent is administered orally.

In another aspect, the invention provides methods for identifying an agent that prevents, reduces, delays or inhibits one or more symptoms of Friedreich's ataxia, comprising contacting a population of cells in vitro with a candidate agent in the presence of an inhibitor of the thioredoxin reductase pathway, wherein an agent that prevents, reduces, delays or inhibits one or more symptoms of Friedreich's ataxia increases cell viability and/or prevents cell death in the presence of the inhibitor of the thioredoxin reductase pathway. The increase in cell viability and/or prevention of cell death can be determined in comparison to a control population of cells that have not been contacted with the candidate agent. The inhibitor of the thioredoxin reductase pathway can be present at a concentration that is lethal or sub-lethal to the population of cells.

In some embodiments, the inhibitor of the thioredoxin reductase pathway is selected from the group consisting of auranofin, and diamide, and mixtures and analogs thereof.

In some embodiments, the methods further comprise the step of selecting for agents that increase viability and/or prevent cell death by at least about 1.4-fold, for example, at least about 1.5-fold, 1.6-fold, 1.7-fold, 1.8-fold, 1.9-fold, 2.0-fold, or more, in comparison to a control population of cells that have not been contacted with the candidate agent.

In some embodiments, the methods further comprise the step of selecting for agents that increase viability and/or prevent cell death with an EC50 concentration of less than about 5 μM, for example, less than about 4 μM, 3 μM, 2 μM, 1 μM, 0.5 μM or less.

In some embodiments, the methods further comprise the step of selecting for agents that increase viability and/or prevent cell death in a dose-dependent manner.

In some embodiments, the candidate agent is a small organic compound, a polypeptide, an antibody or fragment thereof, an amino acid or analog thereof, a carbohydrate, a saccharide or disaccharide, or a polynucleotide.

In some embodiments, the population of cells is a population of fibroblast cells. In some embodiments, the population of cells is a population of neuronal or nerve cells. In some embodiments, the population of cells is a population of dorsal root ganglion cells.

In another aspect, the invention provides methods for preventing, reducing, delaying or inhibiting Friedreich's ataxia in a subject in need thereof comprising administering to the subject an effective amount of an agent identified by the screening methods described herein.

### DEFINITIONS

The terms “individual,” “patient,” “subject” interchangeably refer to a mammal, for example, a human, a non-human primate, a domesticated mammal (e.g., a canine or a feline), an agricultural mammal (e.g., equine, bovine, ovine, porcine), or a laboratory mammal (e.g., rattus, murine, lagomorpha, hamster).

The terms “treating” and “treatment” refer to delaying the onset of, retarding or reversing the progress of, reducing the severity of, or alleviating or preventing either the disease or condition to which the term applies (i.e., Friedreich's ataxia), or one or more symptoms of such disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

The term “effective amount” or “therapeutically effective amount” refers to the amount of an active agent sufficient to induce a desired biological result (e.g., prevention, delay, reduction or inhibition of Friedreich's ataxia). That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount vary with the condition being treated, the stage of advancement of the condition, and the type and concentration of formulation applied.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “frataxin,” “FA,” “X25,” “CyaY” “FARR,” “MGC57199,” “FXN” interchangeably refer to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 300, 400, or more amino acids, or over the full-length, to an amino acid sequence encoded by a frataxin nucleic acid (see, e.g., GenBank Accession Nos. NM_{—}000144.4 (isoform 1); NM_{—}181425.2 (isoform 2); NM_{—}001161706.1 (isoform 3)) or to an amino acid sequence of a frataxin polypeptide (see, e.g. GenBank Accession Nos. NP_{—}000135.2 (isoform 1); NP_{—}852090.1 (isoform 2); NP_{—}001155178.1 (isoform 3)); (2) bind to antibodies, e.g., polyclonal antibodies, raised against an immunogen comprising an amino acid sequence of a frataxin polypeptide (e.g., frataxin polypeptides described herein); or an amino acid sequence encoded by a frataxin nucleic acid (e.g., frataxin polynucleotides described herein), and conservatively modified variants thereof; (3) specifically hybridize under stringent hybridization conditions to an anti-sense strand corresponding to a nucleic acid sequence encoding a frataxin protein, and conservatively modified variants thereof; (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to a frataxin nucleic acid (e.g., frataxin polynucleotides, as described herein, and frataxin polynucleotides that encode frataxin polypeptides, as described herein).

The term “Friedreich's ataxia” and “FRDA” interchangeably to an autosomal recessive congenital ataxia caused by a mutation in gene FXN (formerly known as X25) that codes for frataxin, located on chromosome 9. The genetic basis for FRDA involves GAA trinucleotide repeats in an intron region of the gene encoding frataxin. This segment is normally repeated 5 to 33 times within the FXN gene. In people with Friedreich ataxia, the GAA segment is repeated 66 to more than 1,000 times. People with GAA segments repeated fewer than 300 times tend to have a later appearance of symptoms (after age 25) than those with larger GAA trinucleotide repeats. The presence of these repeats results in reduced transcription and expression of the gene. Frataxin is involved in regulation of mitochondrial iron content. The mutation in the FXN gene causes progressive damage to the nervous system, resulting in symptoms ranging from gait disturbance to speech problems; it can also lead to heart disease and diabetes. The ataxia of Friedreich's ataxia results from the degeneration of nerve tissue in the spinal cord, in particular sensory neurons essential (through connections with the cerebellum) for directing muscle movement of the arms and legs. The spinal cord becomes thinner and nerve cells lose some of their myelin sheath (the insulating covering on some nerve cells that helps conduct nerve impulses). A subject with FRDA may exhibit one or more of the following symptoms: muscle weakness in the arms and legs, loss of coordination, vision impairment, hearing impairment, slurred speech, curvature of the spine (scoliosis), high plantar arches (pes cavus deformity of the foot), carbohydrate intolerance, diabetes mellitus, heart disorders (e.g., atrial fibrillation, tachycardia (fast heart rate) and hypertrophic cardiomyopathy). A subject with FRDA may further exhibit involuntary and/or rapid eye movements, loss of deep tendon reflexes, loss of extensor plantar responses, loss of vibratory and proprioceptive sensation, cardiomegaly, symmetrical hypertrophy, heart murmurs, and heart conduction defects. Pathological analysis may reveal sclerosis and degeneration of dorsal root ganglia, spinocerebellar tracts, lateral corticospinal tracts, and posterior columns

“Administering” refers to local or systemic administration, e.g., including enteral or parenteral administration. Routes of administration for the active agents that find use in the present invention include, e.g., oral (“po”) administration, administration as a suppository, topical contact, intravenous (“iv”), intraperitoneal (“ip”), intramuscular (“im”), intralesional, intranasal, or subcutaneous (“sc”) administration, or the implantation of a slow-release device e.g., a mini-osmotic pump, a depot formulation, and so forth, to a subject. Administration can be by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, ionophoretic and intracranial. Other modes of delivery include, but are not limited to, the use of liposomal formulations, intravenous infusion, transdermal patches, and so forth

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (i.e., other than through the alimentary tract, such as intramuscular, intravenous, intra-arterial, transdermal and subcutaneous) administration.

The term “co-administer” and “co-administering” and variants thereof refer to administration of two active agents proximate in time to one another (e.g., within the same day, or week or period of 30 days, or sufficiently proximate that both drugs can be simultaneously detected in the blood, or otherwise sufficiently proximate that a synergistic effect results from the combined administration). An effect is considered synergistic if a more favorable response and/or fewer side effects are obtained from the co-administration of two (or more) agents than from administration of the same dose of each individual agent as the dose of the combined agent (dose can be measured as moles, moles/kg, mg or mg/kg). For example, co-administration of active agents A and B is considered synergistic if co-administration of 0.5×moles A and 0.5×moles B gives a better efficacy and/or reduced side effects than the separate administration of 1.0×moles A and the separate administration of 1.0×moles B. When co-administered, two or more active agents can be co-formulated as part of the same composition or administered as separate formulations.

The terms “increasing,” “promoting,” “enhancing,” particularly with reference to increasing cell viability and/or preventing cell death, refers to increasing cell viability by a measurable amount using any known method, such as those in the Examples. The cell viability is increased, promoted or enhanced if the number of viable cells in the test cell population is at least about 10%, 20%, 30%, 50%, 80%, or 100% increased, e.g., in comparison to the to a control test population of cells that have not been contacted with an active agent, as described herein. In some embodiments, the cell viability in the test cell population is increased, promoted or enhanced by at least about 1-fold, 2-fold, 3-fold, 4-fold, or more in comparison to a control test population of cells that have not been contacted with an active agent.

The term “candidate agent” refers to any molecule of any composition, including proteins, peptides, nucleic acids, lipids, carbohydrates, organic molecules, inorganic molecules, and/or combinations of molecules which are suspected to be capable of inhibiting a measured parameter (e.g., increased frataxin expression, mitochondrial function, preservation of nerve function) in a treated cell, tissue or subject, e.g., in comparison to an untreated cell, tissue or subject. Likewise any agent determined in a screening assay or otherwise known to have such an activity is referred to as an “active agent” notwithstanding that further preclinical or clinical testing may be needed to show or confirm therapeutic activity. Active agents are sometimes referred to simply as agents or compounds.

As used herein, the phrase “consisting essentially of” refers to the genera or species of active pharmaceutical agents included in a method or composition, as well as any excipients inactive for the intended purpose of the methods or compositions. In some embodiments, the phrase “consisting essentially of” expressly excludes the inclusion of one or more additional active agents other than those expressly recited in the claim.

The term “alkyl,” by itself or as part of another substituent, means, unless otherwise stated, a straight (i.e. unbranched) or branched chain, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene” by itself or as part of another substituent means a divalent radical derived from an alkyl, as exemplified by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms and often 4 or fewer carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′ C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent means a divalent radical derived from a cycloalkyl and heterocycloalkyl, respectively.

Certain agents of the present invention possess asymmetric carbon atoms (optical centers) or double bonds; the racemates, diastereomers, tautomers, geometric isomers and individual isomers are encompassed within the scope of the present invention. The agents of the present invention do not include those which are known to be too unstable to synthesize and/or isolate.

### BRIEF DESCRIPTION OF THE FIGURES

FIG. 1 (top panel) illustrates a pathophysiological model for Friedreich's ataxia based on dorsal root ganglion microarrays and biochemical investigation and drug screening. Frataxin is involved in mitochondrial iron-sulfur cluster biogenesis, and facilitates mitochondrial selenocysteine metabolim, which is essential to the protection of mitochondria from oxidative stress, which is primarily mediated by the selenoenzymes Thioredoxin reductase (Txrd2), and glutathione peroxidase (GPX5). As a result of deficiency of frataxin, these selenoenzymes have decreased activity, and Nrf2 declines, the result is decreased mitochondrial antioxidant protection, increased aggregates, reactive oxygen species, inflammation and neurodegeneration. In addition frataxin interacts with NFS1 of the 2Fe2S cluster biogenesis machinery, necessary for glutaredoxin 2 and ferredoxin 2 function. Reduced function of glutaredoxin 2 and ferredoxin 2 leads to deficiencies of thioredoxin reductase, decreased mitochondrial antioxidant protection, increased aggregates, reactive oxygen species, inflammation and neurodegeneration. In FIG. 1 (bottom panel) we observe that inducers of Nrf2 increase frataxin expression, increase selenocysteine metabolism and Txrd2 and GPX5 activity, and increase iron-sulfur cluster biogenesis, and promote cellular protection.

FIG. 2 illustrates multiple proteins directly or indirectly reduced by thioredoxin reductase are deficient in YG8 mice, and frataxin knockdown reduces thioredoxin reductase activity. DRGs of YG8 mice were microdissected and protein expression of genes measured. FIGS. 2A-C show that the antioxidants Peroxiredoxin-3, Glutaredoxin-1 and Glutathione-S-transferase-1 were each decreased. Glutathione is the most important redox buffer in the cell and low GSH/GSSG indicates increased oxidative stress.

FIG. 2D shows the GSH/GSSG ratio is reduced in FRDA patient lymphoblasts as a result of increased GSSG levels. FIG. 2E shows hemizygous YG8 FRDA mouse model cerebellum had significantly more and about twice the level of GSSG than homozygous mice, causing a decreased GSH/GSSG ratio, demonstrating oxidative stress in this tissue.

FIG. 3 illustrates that multiple proteins reduced in YG8 mice DRGs are reduced by thioredoxin reductase activity, and that frataxin deficiency itself reduces thioredoxin reductase activity, and that frataxin deficiency and inhibition of thioredoxin reductase kill HeLa cells. Frataxin was knocked down using siRNA in HeLa cells and decreased thioredoxin reductase activity was observed (FIG. 3A). Peroxiredoxins, glutaredoxins, thioredoxins, GSSG that were decreased in microarray and Westerns of the YG8 DRGs are ultimately reduced by thioredoxin reductase (FIG. 3C). Frataxin deficiency and thioredoxin reductase deficiency additively caused cell death (FIG. 3B).

FIG. 4 illustrates that DRG neurons with frataxin deficiency died more rapidly when treated with the thioredoxin oxidant diamide, and the thioredoxin reductase inhibitor auranofin (FIG. 4A). This sensitivity was dose-dependent (FIG. 4B) and confirmed in Friedreich's patient fibroblasts (FIG. 4C), and could be reversed by the reductant DTT. The major mitochondrial antioxidant system is thioredoxin reductase (4D). Auranofin is a specific inhibitor of thioredoxin reductase, and diamide is a known oxidizer of thioredoxin. Thus the diamide screen can identify compounds that rescue from thioredoxin reductase deficiency, which include inducers of Nrf2, which are known to induce thioredoxin reductase and other neuroprotective antioxidant functions.

FIG. 5 illustrates that dyclonine induces frataxin expression in FRDA lymphoblasts and HeLa cells. To test if one mechanism of protection from diamide toxicity for dyclonine was an increase in frataxin protein levels, cells were cultured with dyclonine and representative western blots measuring FXN expression are shown for HeLa cells (FIG. 5A) and FRDA lymphoblasts (FIG. 5B). Dyclonine induction of FXN levels was consistent over multiple experiments (FIG. 5C).

FIG. 6 illustrates Dyclonine increases frataxin levels in vivo. To determine the ability of dyclonine to reverse the in vivo FXN protein defect in the YG8 FRDA transgenic mouse model, animals were dosed daily with 1 mg/kg dyclonine via intraperitoneal injection for 6 days and cerebellar and splenocyte frataxin protein level was analyzed. Representative western blot of cerebellum and splenocytes is shown (FIG. 6A) and densitometry of FXN/actin normalization (FIG. 6B).

FIG. 7 illustrates drugs in addition to dyclonine can increase FXN levels in vivo. 20 of the original 40 neuroprotective drugs were shown to increase FXN levels in FRDA patient cells. Of these 20, 8 were tested in the YG8 transgenic mouse model. In addition to dyclonine, dimethyl fumarate, methylene blue, and nifursol were observed to increase frataxin in cerebellum. Representative western blot of cerebellum is shown (FIG. 7B) and densitometry of FXN/actin normalization (FIG. 7A).

FIG. 8 illustrates dimethyl fumarate is a FXN inducer in the YG8 mouse model. To determine ability of dimethyl fumarate to reverse the in vivo FXN protein defect, the YG8 FRDA transgenic mouse model was chosen. Animals were dosed daily with 5 mg/kg dimethyl fumarate via intraperitoneal injection for 6 days and cerebellum and splenocytes examined for FXN levels. Western blot of cerebellum is shown (FIG. 8A) and densitometry of FXN/actin normalization (FIG. 8B) showing dimethyl fumarate induces FXN expression in vivo.

FIG. 9 illustrates Dimethyl fumarate protects from diamide and induces frataxin accumulation in FRDA cells. This protection from diamide toxicity in FRDA cells was dose-dependent (FIG. 9A), and representative blots for FXN expression are shown for HeLa cells (FIG. 9C) and FRDA lymphoblasts (FIG. 9B).

FIG. 10 illustrates phenathiazines protect from diamide and induce frataxin accumulation in FRDA cells. This protection from diamide toxicity was dose dependent (FIG. 10A), and representative blots for FXN expression are shown for FRDA lymphoblasts (FIG. 10B).

FIG. 11 illustrates synergy of identified FXN-inducing drugs. A dose response to dimethyl fumarate in the absence or presence of 5 micromolar dyclonine was determined in FRDA lymphoblasts using the in-cell Western technique (FIG. 11A). To test if Methylene blue also potentiates DMF FXN induction, FRDA patient lymphoblasts were cultured in the presence of 3 micromolar dimethyl fumarate and 3 micromolar methylene blue. Representative blots are shown for HeLa cells (FIG. 11B-C), showing Methylene blue also potentiates DMF FXN induction in vitro.

FIG. 12 illustrates the 20 inducers of frataxin discovered in human cells. Of the original 40 drugs identified as protective in diamide screening assay, 20 were found reproducibly to increase FXN protein levels using traditional western blot or in cell western blot methods.

FIG. 13 illustrates measures the ability of 40 drugs identified as protective by the diamide screening assay to activate the Nrf2(ARE) response element. The activity of 40 drugs to activate the Nrf2 target antioxidant response element was evaluated in a reporter HeLa cell line transduced with ARE-luciferase. Dyclonine, dexamethasone, mepartricin, dimethyl fumarate, tolonium cl, and ebselen increased ARE-luciferase reporter gene expression in HeLa cells (FIG. 13A). ARE induction by dyclonine was dose dependent (FIG. 13B).

FIG. 14 illustrates the Nrf2 protein was deficient in target dorsal root ganglion (DRG) tissue in the available YG8 model of Friedreich's ataxia. DRG tissue was dissected from wild-type, homozygous and hemizygous (affected) mice, protein extracted, and electrophoresed, blotted and quantified. There was a clear deficiency of protective Nrf2 protein in hemizygotes (FIGS. 14 A, B, and C), and the transcriptional targets of Nrf2, i.e. Nqo1 and SOD2, were also decreased (FIGS. 14 B, and C), frataxin expression was significantly correlated with Nrf2 expression (FIG. 14D), frataxin expression was significantly correlated with the Nrf2 target catalase expression.

### DETAILED DESCRIPTION

1. Introduction

Friedreich's ataxia is a neuro- and cardio-degenerative disease, which results from inherited alterations in the frataxin gene decreasing frataxin polypeptide expression. Identification of agents efficacious for the therapy of Friedreich's ataxia has previously been hampered by the availability of relevant and validated, robust high-throughput screens.

The invention is based in part on identification of a new use for several existing agents, that is, for treating Friedreich's ataxia or other neurodegenerative disease. These agents include dyclonine, methylene blue, and dimethylfumarate (DMF). These agents protect cells obtained from Friedreich's ataxia patients from oxidative stress and increase levels of frataxin protein, the hallmark deficiency of Friedreich's ataxia, in a transgenic mouse model of Friedreich's ataxia. It is further shown that each of these agents is an agonist of the Nrf2 pathway. Although an understanding of mechanism, is not essential to practice of the invention, it is believed that the ability of the agents to increase frataxin levels may be the result of any or all of the following mechanism: (a) inhibition of the activity of thioredoxin reductase, to which cells respond by increasing Nrf2 transcription factor, which induces a neuroprotective response, including the induction of frataxin, (b) increasing activity, expression or passage to the nucleus of Nrf2, which induces multiple neuroprotective responses, including the induction of frataxin; (c) increasing mitochondrial iron-sulfur cluster biogenesis which is neuroprotective and results in an increase in frataxin; and (d) increasing histone methylysine transferase, which increases the expression of multiple neuroprotective genes including frataxin.

The present invention is also based, in part, on the discovery of active agents that protect cells isolated from Friedreich's ataxia patients from cell death. Illustrative active agents include inhibitors of the arachidonic acid pathway (e.g., dexamethasone and diphenhydramine and mixtures and analogs thereof); sulfur-containing compounds affecting mitochondria (e.g., lipoic acid, lipoamide, thiamine, and mixtures and analogs thereof); antioxidants (e.g., ebselen, or an analog thereof); inducers of the Nrf2 antioxidant response pathway (e.g., anethole, aspartame, dexamethasone, dimethyl fumarate, dyclonine, ebselen, mepartricin, methylene blue, nifursol, oxfendazole, sulfisoxazole, thioctic acid, tolonium cl, tryptophan/3-hydroxyanthranilate, yohimbine, mixtures and analogs thereof): inducers of the mitochondrial ferredoxin/adrenodoxin pathway (e.g., isoflupredone); and agents that increase the expression levels of frataxin (e.g., anethole, aspartame, cephradine, cotinine, dexamethasone, dimethyl fumarate, diphenhydramine, dyclonine, ebselen, isoflupredone, meclocycline, mepartricin, methylene blue, nifursol, oxfendazole, sulfisoxazole, thioctic acid, tolonium cl, tryptophan/3-hydroxyanthranilate, yohimbine). The active agents find use for preventing, reducing, delaying or inhibiting one or more symptoms of Friedreich's ataxia in a subject in need thereof.

The present invention further provides a relevant high throughput assay for screening for agents useful to treat or ameliorate one or more symptoms of Friedreich's ataxia. Microarray of dorsal root ganglion neurons from the YG8 mouse model of FRDA suggested defects in thiol-related antioxidants, and inhibitors of these antioxidants were tested in Friedreich's patient fibroblasts, which were sensitive to the thioredoxin oxidant diamide and the thioredoxin reductase inhibitor auranofin. Sensitivity to diamide was the specific result of siRNA-mediated frataxin deficiency in a dorsal root ganglion cell line, and could be reversed by DTT and erythropoietin. The cell-based assay was developed for high-throughput screening, e.g., in multiwell plates, with an excellent screening window and low variability, represented by a Z′ value of 0.75 (n=5) and can be used to screen libraries of agents for those that protect Friedreich's patient cells from oxidative (e.g., diamide)-induced death. Active agents that significantly protect Friedreich's cells from thioredoxin oxidation (e.g., by exposure to diamide), in multiple screens can be confirmed by dose-response curves. Active agents of interest also increase frataxin gene expression.

2. Subjects Amenable to Treatment

Patients amenable to treatment include individuals at risk of disease but not showing symptoms, as well as patients presently showing symptoms. Generally, the subject is homozygous for a mutation (a GAA expansion or point mutation) that inhibits or reduces the expression levels of frataxin. For subjects homozygous for a mutation in the frataxin gene that results in insufficient expression levels of the frataxin polypeptide, the risk of developing symptoms of Friedreich's ataxia generally increases with age. Accordingly, in asymptomatic subjects homozygous for a mutation in the frataxin gene that results in insufficient expression levels of the frataxin polypeptide, in certain embodiments, prophylactic application is contemplated for subjects over 5 years of age, for example, in subjects over about 6, 7, 8, 9, 10, 11, 12, 13, 14, 15, 16, 17, 18, 19, or 20 years of age. Subjects with late or very late onset of disease, as described above can also be treated.

The present methods are especially useful for individuals who do have a known genetic risk of Friedreich's ataxia, whether they are asymptomatic or showing symptoms of disease. Such individuals include those having relatives who have experienced this disease (e.g., a parent, a grandparent, a sibling), and those whose risk is determined by analysis of genetic and/or biochemical markers. Genetic markers of risk toward Friedreich's ataxia include mutations in the frataxin gene, in humans located on chromosome 9, in various embodiments mapped to an intron at 9q13-q21.

In some embodiments, the subject is asymptomatic but has familial and/or genetic risk factors for developing Friedreich's ataxia. In asymptomatic patients, treatment can begin at any age (e.g., 5, 6, 7, 8, 9, 10, 11, 12, 13, 14, 15, 16, 17, 18, 19, or 20 years of age, or older).

In some embodiments, the subject is exhibiting symptoms of Friedreich's ataxia, for example, muscle weakness in the arms and legs, loss of coordination, loss of deep tendon reflexes, loss of extensor plantar responses, loss of vibratory and proprioceptive sensation, vision impairment, involuntary and/or rapid eye movements, hearing impairment, slurred speech, curvature of the spine (scoliosis), high plantar arches (pes cavus deformity of the foot), carbohydrate intolerance, diabetes mellitus, and heart disorders (e.g., atrial fibrillation, tachycardia (fast heart rate), hypertrophic cardiomyopathy, cardiomegaly, symmetrical hypertrophy, heart murmurs, and heart conduction defects).

In some embodiments, the subject does not suffer from a disease condition other than Friedreich's ataxia. For example, the subject does not suffer from a disease condition other than Friedreich's ataxia that can be or is oftentimes treated by the active agent.

In some embodiments, the subject does not have or is not diagnosed with diabetes. Some subjects lack neurodegenerative diseases other than Friedreich's ataxia. Some subjects lack sore throats or diseases other than Friedreich's ataxia known to be treatable by dyclonine.

### Active Agents

Active agents that find use in the present methods are effective in preventing, reducing, delaying or inhibiting one or more symptoms of Friedreich's ataxia. In various embodiments, agents that find use directly or indirectly (e.g., via the NRF2 pathway) induce or increase expression of frataxin polypeptide from the frataxin gene, increase mitochondrial function in the cells of a subject with Friedreich's ataxia, and/or increase cell viability and/or prevent cell death in a subject with Friedreich's ataxia.

Preferred agents include dyclonine, methylene blue and DMF and analogs thereof having similar activity including ability to cross the blood brain barrier in sufficient amount to exert a therapeutic or prophylactic effect

Dimethyl fumarate and analogs thereof include compounds conforming to formula (I):

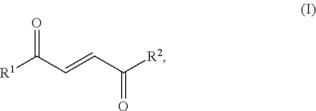

or a pharmaceutically acceptable salt thereof; wherein R¹ and R² are independently selected from —CH₃₋ₙEₙ, OH, O⁻, and (C₁₋₈) alkoxy (branched or unbranched), provided that at least one of R¹ and R² is (C₁₋₈) alkoxy. It is also to be understood that the present invention is considered to include cis and trans isomers, stereoisomers as well as optical isomers, e.g. mixtures of enantiomers as well as individual enantiomers and diastereomers, which arise as a consequence of structural asymmetry in selected compounds of the present series. Formula I compounds include trans (fumarate) and cis (maleate) isomers. E is an electron withdrawing group. Examples of electron withdrawing groups include —NO₂, —N(R₂), —N(R₃)⁺, —N(H₃)⁺, —SO₃H, —SO₃R′, —S(O₂)R′ (sulfone), —S(O)R′ (sulfoxide), —S(O₂)NH₂ (sulfonamide), —SO₂NHR′, —SO₂NR′₂, —PO(OR′)₂, —PO₃H₂, —PO(NR′₂)₂, pyridinyl (2-, 3-, 4-), pyrazolyl, indazolyl, imidazolyl, thiazolyl, benzothiazolyl, oxazolyl, benzimidazolyl, benzoxazolyl, isoxazolyl, benzisoxazolyl, triazolyl, benzotriazolyl, quinolinyl, isoquinolinyl, quinazolinyl, pyrimidinyl, a 5 or 6-membered heteroaryl with a C—N double bond optionally fused to a 5 or 6 membered heteroaryl, pyridinyl N-oxide, —C≡N, —CX′₃, —C(O)X′, —COOH, —COOR′, —C(O)R′, —C(O)NH₂, —C(O)NHR′, —C(O)NR′₂, —C(O)H, —P(O)(OR′)OR″ and X′, wherein X′ is independently halogen (e.g. F, Cl, Br, I) and R, R′ and R″ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or similar Substituents (e.g. a substituent group, a size limited substituent group or a lower substituent group). Examples of dimethyl fumarate analogs include but are not restricted to monomethyl fumarate (MMF), monomethyl maleate, monoethyl fumarate, monoethyl maleate, monobutyl fumarate, monobutyl maleate, monooctyl fumarate, monoctyl maleate, mono(phenylmethyl)fumarate, mono(phenylmethyl)maleate, mono(2-hydroxypropyl)fumarate, mono(2-hydroxypropyl)maleate, mono(2-ethylhexyl)fumarate, mono(2-ethylhexyl)maleate, dimethylfumarate, dimethyl maleate, diethyl fumarate, diethyl maleate, dipropyl fumarate, dipropyl maleate, diisopropyl fumarate, diisopropyl maleate, dibutyl fumarate, dibutyl maleate, diisobutyl fumarate, diisobutyl maleate, diheptyl fumarate, diheptyl maleate, bis(2-ethylhexyl)fumarate, bis(2-ethylhexyl)maleate, (−)-Dimenthyl fumarate, (−)-Bis((S)-1-(ethoxycarbonyl)ethyl)fumarate, (−)-Bis((S)-1-(ethoxycarbonyl)ethyl)maleate, Bis(2-trifluoroethyl)fumarate, Bis(2-trifluoroethyl)maleate.

Dyclonine and an analogs thereof include compounds conforming to formula (II):

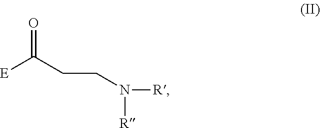

wherein E is substituted or unsubstituted aryl, or substituted or unsubstituted heteroaryl; and R′ and R″ taken together with the N to which each is bound form a primary, secondary or tertiary amine, or together with the N to which each is bound, R′ and R″ form a cyclic amine group (e.g., a

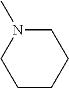

group). For example, E can be substituted with R³ to form an R³-substituted C₍₆₋₁₀₎ aryl, or R³-substituted C₍₂₋₉₎ heteroaryl. R³ can be a substituent on any available position of the aryl or heteroaryl ring. R′ and R″ taken together with the N to which each is bound can be a 3-membered cyclic aziridines, 4-membered cyclic azetidines, 5-membered cyclic pyrrolidines, 6-membered cyclic piperidines, 7-membered cyclic azepanes, 8-membered cyclic azocanes. Specific examples of dyclonine analogs include

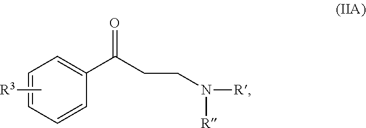

wherein R³, R′ and R″ are as described above. More preferred are the compounds of formula (IIB):

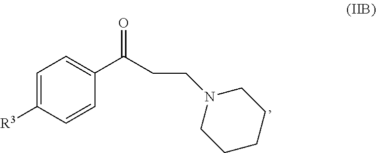

wherein R³ is hydrogen, halogen (F, Cl, Br, I), —CN, —OH, —NH₂, —COOH, —CF₃, (C₍₂₋₈₎ alkoxy), such as, —OCH₃, —OC₂H₅, —OC₃H₇, —OC₄H₉, —OC₅H₁₁—OCOCH₃, R⁴-substituted C₍₁₋₈₎ alkyl, unsubstituted C₍₁₋₈₎ alkyl, R⁴-substituted C₍₁₋₈₎ heteroalkyl, unsubstituted C₍₁₋₈₎ heteroalkyl, R⁴-substituted C₍₃₋₇₎ cycloalkyl, unsubstituted C₍₃₋₇₎ cycloalkyl, R⁴-substituted C₍₂₋₇₎ heterocycloalkyl, unsubstituted C₍₂₋₇₎ heterocycloalkyl, R⁴-substituted C₍₆₋₁₀₎ aryl, unsubstituted C₍₆₋₁₀₎ aryl, R⁴-substituted C₍₂₋₉₎ heteroaryl or unsubstituted C₍₂₋₉₎ heteroaryl, or a pharmaceutically acceptable salt thereof, wherein R⁴ is in each instance selected from the group consisting of halogen (F, Cl, Br, I), —CN, —OH, —NH₂, —COOH, —CF₃, —OCH₃, —OC₂H₅, —OC₃H₇, and —OCOCH₃.

Analogs of methylene blue include leuco-methylene blue and acetyl-methylene blue, 2-chlorophenothiazine, phenothiazine, toluidine blue, tolonium chloride, toluidine blue O, seleno toluidine blue, methylene green, chlorpromazine, sulphoxide chlorpromazine, sulphone chlorpromazine, chlordiethazine promethazine, thioproperazine, prochlorperazine, pipotiazine, dimetotiazine, propericiazine, metazionic acid, oxomemazine neutral red, iminostilbene, and imipramine, or a pharmaceutically acceptable salt thereof. Methylene blue and analogs thereof also include compounds conforming to formula (III):

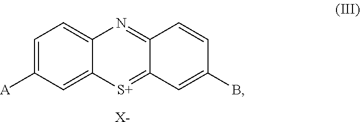

wherein A and B are independently selected from hydrogen, halogen (F, Cl, Br, I), —CN, —OH, —NH₂, —COOH, —CF₃, —OCH3, —OC2H5, —OC3H7, —OCOCH3, or

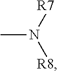

wherein R⁷ and R⁸ are each independently H, OCOCH3, or linear or branched CₙH₂ₙY, wherein n is 1-6, Y is H, F, Cl, Br, I, OH, OCH3, OC2H5, OC3H7, CN, or OCOCH3. X— is a counteranion. Examples of counteranions include Cl⁻, Br⁻, I⁻, F⁻, NO₃⁻, HSO₄⁻, CH₃CO₂⁻, or a dianion such as SO₄²⁻, HPO₄²⁻, or a trianion such as PO₄³⁻. Examples of R⁷ and R⁸ include n-propyl, n-butyl, or n-pentyl.

Methylene blue analogs also include compounds conforming to formula (IV):

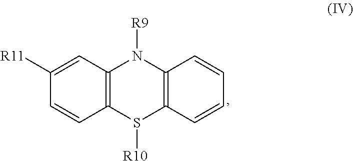

(S atom can be neutral or positively charged) wherein R⁹ can be

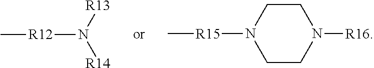

R¹³, R¹⁴ and R¹⁶ are each independently hydrogen, substituted or unsubstituted alkyl, —OH, and —R¹⁷—OH. R¹², R¹⁵ and R¹⁷ are each independently substituted or unsubstituted alkylene.
For example, R⁹ can be —CH₂N(CH₃)₂, —CH₂CH(CH₃)CH₂N(CH₃)₂, —CH₂C(CH₃)₂CH₂N(CH₃)₂, —CH₂CH(CH₃)CH₂N(C₂H₅)₂, —CH₂CH(CH₃)N(C₂H₅)₂, —(CH₂)₂N(C₂H₅)₂, —(CH₂)₃N(CH₃)₂, —CH₂CH(CH₃)N(CH₃)₂, —CH₂CH(CH₃) CH₂N(CH₃)₂, —CH₂C(CH₃)₂CH₂N(CH₃)₂, —CH₂CH(CH₃)CH₂N(C₂H₅)₂, —CH₂CH(CH₃)N(C₂H₅)₂, —(CH₂)₂N(C₂H₅)₂,

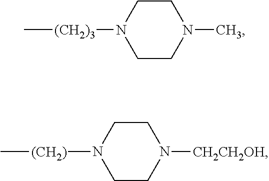

### —CH₂CH(CH₃)N(CH₃)₂,

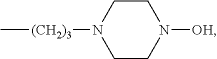

—CH₃, —(CH₂)₃N(CH₃)₂, (CH₂)₃N(CH₃)₂, —CH₂CH(CH₃)CH₂N(CH₃)₂. R¹⁰ can be absent or present. If present, R¹⁰ is —OH or ═O. R¹¹ can be hydrogen, halogen (F, Cl, Br, I), —CN, —CF₃, —CH₂CO₂H, —SO₂N(CH₃)₂.

In various embodiments, the active agents for use in treating, mitigating or preventing one or more symptoms of Friedreich's ataxia include inhibitors of the arachidonic acid pathway (e.g., (e.g., dexamethasone and diphenhydramine and mixtures and/or analogs and/or pharmaceutically acceptable salts thereof); sulfur-containing compounds affecting mitochondria (e.g., (e.g., lipoic acid, thioctic acid, lipoamide, thiamine, and/or analogs and/or pharmaceutically acceptable salts thereof); antioxidants (e.g., ebselen, or an analog and/or a pharmaceutically acceptable salt thereof); inducers of the Nrf2 antioxidant response pathway (e.g., anethole, aspartame, dexamethasone, dimethyl fumarate, dyclonine, ebselen, mepartricin, methylene blue, nifursol, oxfendazole, sulfisoxazole, thioctic acid, tolonium cl, tryptophan/3-hydroxyanthranilate, yohimbine, and mixtures and/or analogs and/or pharmaceutically acceptable salts thereof): inducers of the mitochondrial ferredoxin/adrenodoxin pathway (e.g., isoflupredone)); and agents that increase the expression levels of frataxin (e.g., anethole, aspartame, cephradine, cotinine, dexamethasone, dimethyl fumarate, diphenhydramine, dyclonine, ebselen, isoflupredone, meclocycline, mepartricin, methylene blue, nifursol, oxfendazole, sulfisoxazole, thioctic acid, tolonium cl, tryptophan/3-hydroxyanthranilate and mixtures and/or analogs and/or pharmaceutically acceptable salts thereof).

In some embodiments, the active agent for use in treating, mitigating or preventing one or more symptoms of Friedreich's ataxia is any of anethole, aspartame, cephradine, cotinine, dexamethasone, dimethyl fumarate, diphenhydramine, dyclonine, ebselen, isoflupredone, meclocycline, mepartricin, methylene blue, nifursol, oxfendazole, sulfisoxazole, thioctic acid, tolonium cl, tryptophan/3-hydroxyanthranilate, yohimbine and mixtures and/or analogs and/or pharmaceutically acceptable salts thereof.

In some embodiments, the active agent does not disrupt the cytoskeleton or microtubules in a cell. In some embodiments, the active agent is not an azole, e.g., is not selected from the group consisting of nocodazole, albendazole, fenbendazole, oxfendazole, oxibendazole, methiazole, parbendazole, or any derivatives, metabolites, or analogs thereof. In some embodiments, the active agent is not a cytochalasin, a derivative, metabolite, or analog thereof.

Further agents of use can be identified using the screening methods described herein.

3. Methods of Treatment and Prevention

In various methods of treatment, the subject may already exhibit symptoms of disease or be diagnosed as having disease. For example, the subject may exhibit symptoms of Friedreich's ataxia or be diagnosed as having Friedreich's ataxia. In such cases, administration of one or more active agents described herein and/or analogs and/or pharmaceutically acceptable salts thereof can reverse or delay progression of and or reduce the severity of disease symptoms.

The effectiveness of treatment can be determined by comparing a baseline measure of a parameter of disease before administration of the one or more active agents described herein and/or analogs and/or pharmaceutically acceptable salts thereof is commenced to the same parameter one or more timepoints after the one or more active agents described herein and/or analogs and/or pharmaceutically acceptable salts thereof has been administered. The parameter of disease can be one or more of the signs or symptoms of Friedreich's ataxia (or other neurodegenerative disease) described herein. Measurement of a level of frataxin, particularly in the blood (e.g., in PBMC's), is a preferred biomarker, an increase in level responsive to treatment being an indication that treatment is effective.

For the purposes of prophylaxis, the subject may be asymptomatic, but have one or more genetic risk factors, as described herein, and/or be of a defined threshold age. Subjects may also be asymptomatic but judged to be at high risk for Friedreich's ataxia based on genetic tests, or other predictive tests. Alternatively, the subject may be exhibiting symptoms of early stages of disease. In such cases, administration of one or more active agents described herein and/or analogs and/or pharmaceutically acceptable salts thereof can prevent or delay onset of disease or progression of Friedreich's ataxia (or other neurodegenerative disease) into later stages of disease, and/or reduce the severity of the disease once present.

Measurable parameters for evaluating the effectiveness of the prevention regime are as discussed herein for therapy and monitoring.

4. Formulation and Administration of Active Agents

a. Formulation

The one or more active agents described herein and/or analogs and/or pharmaceutically acceptable salts thereof can be administered orally, parenterally, (intravenously (IV), intramuscularly (IM), depo-IM, subcutaneously (SQ), and depo-SQ), sublingually, intranasally (e.g., inhalation, nasal mist or drops), intrathecally, topically, transmucosally, bucally, sublingually, ionophoretically or rectally.

Compositions are provided that contain therapeutically effective amounts of the one or more active agents. The compounds are preferably formulated into suitable pharmaceutical preparations such as tablets, capsules, or elixirs for oral administration or in sterile solutions or suspensions for parenteral administration.

The one or more active agents described herein and/or analogs and/or pharmaceutically acceptable salts thereof can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures described, for example, by March (1992) Advanced Organic Chemistry; Reactions, Mechanisms and Structure, 4th Ed. N.Y. Wiley-Interscience. Prodrugs of the agents readily undergo chemical changes under physiological conditions to provide the agents of the present invention. Conversion usually occurs after administration to a patient.

Methods of formulating such derivatives are known. For example, the disulfide salts of a number of delivery agents are described in WO 2000/059863 which is incorporated herein by reference. Similarly, acid salts of agents can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, carboxylic acid, propionic acid, glycolic acid, pyruvic acid, oxalic acid, malic acid, malonic acid, succinic acid, suberic acid, lactic acid, benzene sulfonic acid, p-tolylsulfonic acid, arginine, glucuronic acid, galactunoric acid phthalic acid, maleic acid, fumaric acid, tartaric acid, citric acid, benzoic acid, cinnamic acid isobutyric, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like (see, e.g., Berge et al., J. Pharm. Sci. 66, 1-19 (1977).

Although dyclonine has usually been supplied in the form of an HCl salt, acid salts with weaker acids (e.g., pKa 1-6-9 or preferably pKa 4-6.5) are preferred for parenteral administration. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHmax to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, besylate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like. Suitable cationic salt forms include, but are not limited to aluminum, benzathine, calcium, ethylene diamine, lysine, magnesium, meglumine, potassium, procaine, sodium, tromethamine, zinc, and the like.

In various embodiments, preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques described in the pertinent literature. For example, amides may be prepared from esters, using suitable amine reactants, or they may be prepared from an anhydride or an acid chloride by reaction with ammonia or a lower alkyl amine.

About 1 to 1000 mg of a compound or mixture of the one or more active agents or a physiologically acceptable salt or ester is compounded with a physiologically acceptable vehicle, carrier, excipient, binder, preservative, stabilizer, flavor, and so forth, in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 1-1000 mg, 2-800 mg, 5-500 mg, 10-400 mg, 50-200 mg, e.g., about 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 200 mg, 300 mg, 400 mg, 500 mg, 600 mg, 700 mg, 800 mg, 900 mg or 1000 mg of the active ingredient. The term “unit dosage from” refers to physically discrete units suitable as unitary (i.e., single) dosages for human subjects and other mammals, each unit containing a predetermined quantity of active material calculated to produce the desired therapeutic effect, in association with a suitable pharmaceutical excipient.

To prepare compositions, the one or more active agents is mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined

Pharmaceutical carriers or vehicles suitable for administration of the compounds provided herein include any such carriers known to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods include, but are not limited to, using cosolvents such as dimethylsulfoxide (DMSO), using surfactants such as Tween™, and dissolution in aqueous sodium bicarbonate. Derivatives of the compounds, such as salts or prodrugs may also be used in formulating effective pharmaceutical compositions.

The concentration of the one or more active agents is effective for delivery of an amount upon administration that lessens or ameliorates at least one symptom of the disorder for which the compound is administered and/or that is effective in a prophylactic context. Typically, the compositions are formulated for single dosage (e.g., daily) administration.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder. A therapeutically or prophylactically effective dose can be determined by first administering a low dose, and then incrementally increasing until a dose is reached that achieves the desired effect with minimal or no undesired side effects.

In various embodiments, one or more active agents described herein and/or analogs and/or pharmaceutically acceptable salts thereof can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the one or more active agents. The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, medipads, creams, and the like for topical or transdermal administration.

The concentration and/or amount of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. The precise dosage and duration of treatment is a function of the disease being treated and may be determined empirically using known testing protocols or by extrapolation from in vivo or in vitro test data. Concentrations and dosage values may also vary with the severity of the condition to be alleviated. For any particular subject, specific dosage regimens can be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound can be provided in a formulation that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

In various embodiments, the tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, medicated chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Suitable carriers for intravenous administration include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The one or more active agents described herein and/or analogs and/or pharmaceutically acceptable salts thereof may be prepared with carriers that protect them against rapid elimination from the body, such as time-release formulations or coatings. Controlled release is a mechanism of formulation to release a drug over an extended time. Use of controlled release formulation may reduce the frequency of administration, reduce fluctuations in blood concentration and protect the gastrointestinal tract from side effects. For example, the anesthetic effect of dyclonine on the mouth and sore throat, which underlies its traditional use in treating sore throats, can be reduce by use of a controlled release formulation. The active compounds may be prepared with carriers that protect the compound against rapid elimination from the body, such as time-release formulations or coating. Such carriers include controlled release formulations (also known as modified, delayed, extended or sustained release or gastric retention dosage forms, such as the Depomed GR™ system in which agents are encapsulated by polymers that swell in the stomach and are retained for about eight hours, sufficient for daily dosing of many drugs). Controlled release systems include microencapsulated delivery systems, implants and biodegradable, biocompatible polymers such as collagen, ethylene vinyl acetate, polyanhydrides, polyglycolic acid, polyorthoesters, polylactic acid, matrix controlled release devices, osmotic controlled release devices, multiparticulate controlled release devices, ion-exchange resins, enteric coatings, multilayered coatings, microspheres, liposomes, and combinations thereof. The release rate of the active ingredient can also be modified by varying the particle size of the active ingredient(s). Examples of modified release include, e.g., those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

b. Route of Administration and Dosing

In various embodiments, the one or more active agents described herein and/or analogs and/or pharmaceutically acceptable salts thereof can be administered orally, parenterally (IV, IM, depo-IM, SQ, and depo-SQ), sublingually, intranasally (inhalation), intraspinally, intrathecally, topically, or rectally. Dosages of agents that are known for prior use to treat or prevent a disease condition other than Friedreich's ataxia may provide a starting point for the purpose of ameliorating the symptoms of Friedreich's ataxia. However, higher dosages of some agents are preferable for treating Friedreich's ataxia than existing indications as is the case for dyclonine.

In various embodiments, the one or more active agents described herein and/or analogs and/or pharmaceutically acceptable salts thereof may be administered enterally or parenterally. Oral formulations include tablets and capsules as well as liquid dosage forms such as solutions, suspensions, and elixirs. When the solid dosage forms are used, it is preferred that they be of the sustained release type so that the one or more active agents need to be administered only once or twice daily (or less frequency).

The oral dosage forms can be administered to the patient 1, 2, 3, or 4 times daily or less frequently, such as on alternate days, every third day, twice a week or once a week. It is preferred that the one or more active agents be administered either three or fewer times, more preferably once or twice daily. Oral dosage forms are preferably designed so as to protect the one or more active agents from the acidic environment of the stomach, such as by enteric coated or by use of capsules filled with small spheres each coated to protect from the acidic stomach.

When administered orally, an administered amount therapeutically effective to prevent, mitigate or treat Friedreich's ataxia is from about 0.1 mg/day to about 200 mg/day, for example, from about 1 mg/day to about 100 mg/day, for example, from about 5 mg/day to about 50 mg/day. In some embodiments, the subject is administered the one or more active agents at a dose of about 0.05 to about 0.50 mg/kg or 0.1 mg/kg-10 mg/kg or 0.5 mg/kg to 5 mg/kg, for example, about 0.05 mg/kg, 0.10 mg/kg, 0.20 mg/kg, 0.33 mg/kg, 0.50 mg/kg, 1 mg/kg, 5 mg/kg or 10 mg/kg. Although a patient may be started at one dose, that dose may be varied (increased or decreased, as appropriate) over time as the patient's condition changes. Depending on outcome evaluations, higher doses may be used. For example, in certain embodiments, up to as much as 1000 mg/day can be administered, e.g., 200 mg/day, 300 mg/day, 400 mg/day, 500 mg/day, 600 mg/day, 700 mg/day, 800 mg/day, 900 mg/day or 1000 mg/day.

The one or more active agents described herein and/or analogs and/or pharmaceutically acceptable salts thereof may also be advantageously delivered in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

In various embodiments, the one or more active agents and/or analogs thereof can be administered parenterally, for example, by IV, IM, depo-IM, SC, or depo-SC. When administered parenterally, a therapeutically effective amount of about 0.5 to about 1000 mg/day, preferably from about 5 to about 500 or 50-200 mg daily should be delivered. In various embodiments, the parenteral dosage form is a depo formulation in which case a larger amount of drug can be administered with reduced frequency.

In various embodiments, the one or more active agents and/or analogs thereof can be administered sublingually. When given sublingually, the one or more active agents and/or analogs thereof can be given one to four times daily in the amounts described above for IM administration.

In various embodiments, the one or more active agents and/or analogs thereof can be administered intranasally. Appropriate formulations include a nasal spray or dry powder. The dosage of the one or more active agents and/or analogs thereof for intranasal administration is the amount described above for IM administration.

In various embodiments, the one or more active agents and/or analogs thereof can be administered intrathecally in a parenteral formulation. The dosage of the one or more active agents and/or analogs thereof for intrathecal administration is the amount described above for IM administration.

In certain embodiments, the one or more active agents and/or analogs thereof can be administered topically or transdermally. When given by this route, the appropriate dosage form is a cream, ointment, or patch. When administered topically, the dosage can be from about 0.5 mg/day to about 200 mg/day. Because the amount that can be delivered by a patch is limited, two or more patches may be used. The number and size of the patch is not important, what is important is that a therapeutically effective amount of the one or more active agents and/or analogs thereof be delivered. The one or more active agents and/or analogs thereof can be administered rectally by suppository. When administered by suppository, the therapeutically effective amount can be from about 0.5 mg to about 500 mg.

In various embodiments, the one or more active agents and/or analogs thereof can be administered by implants. When administering one or more active agents by implant, the therapeutically effective amount is the amount described above for depot administration.

The exact dosage and frequency of administration depends on the particular condition being treated (e.g., whether Friedreich's ataxia or other neurodegenerative disease described below), the severity of the condition being treated, the age, weight, general physical condition of the particular patient, and other medication the individual may be taking.

Exemplary daily dosages of dyclonine range from 1-1000 mg per patient, for example, 30-500, 50-200 mg or 75-150 mg. Exemplary dosages on a per kg base range from 0.1 to 10 mg/kg, for example 1-10 mg/kg, 0.5-5 mg/kg or 0.5, 1, 1.5, 2, 3 or 5 mg/kg per day. In some methods, the dose is at least 50 mg or at least 100 mg per day. In some methods, the dose is at least 0.1, 0.5 or 1.0 mg/kg. Dyclonine is often supplied in the form of dyclonine HCl (e.g., as a 0.5% or 1.0% topical solution from AstraZeneca). However, as mentioned above, other acid salts are preferred for injectable formulations. Preferred formulations include oral, transmucosal (e.g., a mouse wash, chewing gum, or oral gel), buccal and parenteral (e.g., suitable for intravenous, intramuscular or subcutaneous injection). Controlled release and particularly gastric release formulations are preferred.

5. Combination Therapies

The one or more active agents described herein and/or analogs thereof can be used in combination with each other or with other therapeutic agents or approaches used to treat, mitigate or prevent Friedreich's ataxia. For example, the one or more active agents described herein and/or analogs thereof can be co-administered with a histone deacetylase (HDAC) inhibitor. Preferred combinations include dyclonine (or an analog thereof) with DMF (or an analog thereof) and/or methylene blue (or an analog thereof). DMF and methylene blue can also be used in combination. Preferably combinations act synergistically.

6. The Nrf2 Pathway

Nuclear factor (erythroid-derived 2)-like 2, also known as Nrf2, is a transcription factor that in humans is encoded by the NFE2L2 gene. Under normal conditions, Nrf2 is tethered in the cytoplasm by another protein called Kelch like-ECH-associated protein 1 (Keap1). Keap1 acts as a substrate adaptor protein for Cullin 3-based ubiquitination, which results in the proteasomal degradation of Nrf2. Oxidative stress or electrophilic stress disrupts critical cysteine residues in Keap1, resulting in a disruption of the Keap1-Cul3 ubiquitination system and a build-up of Nrf2 in the cytoplasm. Unbound Nrf2 is then able to translocate into the nucleus, where it heterodimerizes with a small Maf protein and binds to an Antioxidant Response Element (ARE) in the upstream promoter region of many anti-oxidative genes to initiate transcription of many cytoprotective proteins. These include NAD(P)H quinone oxidoreductase, glutamate-cysteine ligase, Heme oxygenase-1 (HMOX1, HO-1), the glutathione S-transferase (GST) family, the UDP-glucuronosyltransferase (UGT) family, thioredoxin reductase and multidrug resistance-associated proteins. An Nrf2 agonist means an agent that increases the level of Nrf2 protein, or its activity, or its translocation to the nucleus thereby resulting in increased expression of one or more gene subjective to activation by Nrf2.

7. Other Indications and Agents

Active agents determined to have activities in agonizing the NRF2 pathway and inducing frataxin (e.g., dyclonine, methylene blue, DMF and their analogs) can also be used for treatment or prophylaxis of other diseases associated with less than optimal activity of the NRF2 pathway. Agonizing the Nrf2 pathway also provides relief from inflammatory degenerative conditions including neurodegenerative disease. Such diseases include multiple neurodegenerative diseases, such as Alzheimer's disease, Parkinson's disease, Huntington's disease, ALS, and stroke. A transgenic mouse model suitable for screening for Alzheimer's disease is a triple transgenic mouse containing mutated presenilin, tau and amyloid precursor protein transgenes (see, e.g., U.S. Pat. No. 7,479,579). A transgenic model of Parkinson's disease including an alpha synuclein transgene is described by Masliah et al., Neuron. 2005; 46(6):857-68. Mouse models of Huntington's disease are disclosed by Beal et al., Nature Reviews Neuroscience 5, 373-384 (May 2004). Transgenic mice with a SOD1 mutation can be used in screening agents for activity against ALS and are available from the Jackson Laboratory. Another ALS model has a TDP-43 transgene (Wils, PNAS 2010 vol. 107, 3858-3863). Effects of agent on stroke can be assessed in rats subject to cerebral ischemia (see e.g., U.S. Pat. No. 7,595,297). Other examples of neurodegenerative diseases include conditions characterized by neurodegeneration and/or neuroinflammation, i.e., a condition in which either or both of those processes leads to a failure of the subjects' nervous system to function normally. The loss of normal function may be located in either or both of the central nervous system (e.g., the brain, spinal cord) and the peripheral nervous system. Examples of such conditions include Adrenal Leukodystrophy (ALD), Alcoholism, Alexander's disease, Alper's disease, Ataxia telangiectasia, Batten disease (also known as Spielmeyer-Vogt-Sjδgren-Batten disease), Bovine spongiform encephalopathy (BSE), Canavan disease, Cerebral palsy, Cockayne syndrome, Corticobasal degeneration, Creutzfeldt-Jakob disease, Familial Fatal Insomnia, Frontotemporal lobar degeneration, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjogren-Batten disease (also known as Batten disease), Spinocerebellar ataxia, Spinal muscular atrophy, Steele-Richardson-Olszewski disease, Tabes dorsalis, Toxic encephalopathy, LHON (Leber's Hereditary optic neuropathy), MELAS (Mitochondrial Encephalomyopathy; Lactic Acidosis; Stroke), MERRF (Myoclonic Epilepsy; Ragged Red Fibers), PEO (Progressive External Opthalmoplegia), Leigh's Syndrome, MNGIE (Myopathy and external ophthalmoplegia; Neuropathy; Gastro-Intestinal; Encephalopathy), Kearns-Sayre Syndrome (KSS), NARP, Hereditary Spastic Paraparesis, Mitochondrial myopathy. Lung disease including asthma is often inflammatory, and induction of Nrf2 can be protective. In neurodegenerative diseases, agonizing the NRF2 pathway reduces inflammation of microglia that attach neurons, that may have suffered amyloidogenic deposits, or stroke-mediated damage. The NRF2 pathway is not necessarily suppressed in such individuals. However, agonizing the cellular pathway beyond normal levels can be useful providing a defense mechanism against oxidative stress or inflammation. In contrast to Friedreich's ataxia, these diseases are not characterized by frataxin deficiency. However, an increased level of frataxin protein in response to treatment can still be useful as a biomarker indicating a positive response to treatment. As in other methods, such a level is preferably measured in the blood, such as in PBMC's.

All disclosure of the application (for example, dosages, routes of administration, formulations) for treating Friedreich's ataxia also applies mutatis mutandis to treatment of other neurodegenerative diseases.

Additional agents having activities shown for dyclonine, methylene blue or DMF in agonizing NRF2 and inducing frataxin protein expression can also be used for treatment or prophylaxis of Friedreich's ataxia. Such agents can be identified, for example, by performing screening methods described below.

8. Monitoring Efficacy

Clinical efficacy can be monitored using biomarkers among other methods. Measurable biomarkers to monitor efficacy include, but are not limited to, monitoring one or more of the physical symptoms of Friedreich's ataxia, including muscle weakness in the arms and legs, loss of coordination, loss of deep tendon reflexes, loss of extensor plantar responses, loss of vibratory and proprioceptive sensation, vision impairment, involuntary and/or rapid eye movements, hearing impairment, slurred speech, curvature of the spine (scoliosis), high plantar arches (pes cavus deformity of the foot), carbohydrate intolerance, diabetes mellitus, and heart disorders (e.g., atrial fibrillation, tachycardia (fast heart rate), hypertrophic cardiomyopathy, cardiomegaly, symmetrical hypertrophy, heart murmurs, and heart conduction defects). Observation of the stabilization, improvement and/or reversal of one or more symptoms indicates that the treatment or prevention regime is efficacious. Observation of the progression, increase or exacerbation of one or more symptoms indicates that the treatment or prevention regime is not efficacious. A preferred biomarker for assessing treatment in Friedreich's ataxia is a level of frataxin. This marker is preferably assessed at the protein level, but measurement of mRNA encoding frataxin can also be used as a surrogate measure of frataxin expression. Such a level can be measured in a blood sample, preferably on PBMC's. Such a level is reduced in subjects with Friedreich's ataxia relative to a control population of undiseased individuals. Therefore, an increase in level provides an indication of a favorable treatment response, whereas an unchanged or decreasing levels provides an indication of unfavorable or at least non-optimal treatment response.

Efficacy can also be determined by determining the level of sclerosis and/or degeneration of dorsal root ganglia, spinocerebellar tracts, lateral corticospinal tracts, and posterior columns. This may be accomplishing using medical imaging techniques, e.g., magnetic resonance imaging or tomography techniques, e.g., computed tomography (CT) scan or computerized axial tomography (CAT) scan. Subjects who maintain the same level or a reversal of sclerosis and/or degeneration indicate that the treatment or prevention regime is efficacious. Conversely, subjects who show a higher level or a progression of sclerosis and/or degeneration indicate that the treatment or prevention regime has not been efficacious.

In certain embodiments, the monitoring methods can entail determining a baseline value of a measurable biomarker or disease parameter in a subject before administering a dosage of the one or more active agents described herein, and comparing this with a value for the same measurable biomarker or parameter after a course of treatment.

In other methods, a control value (i.e., a mean and standard deviation) of the measurable biomarker or parameter is determined for a control population. In certain embodiments, the individuals in the control population have not received prior treatment and do not have Friedreich's ataxia, nor are at risk of developing Friedreich's ataxia. In such cases, if the value of the measurable biomarker or clinical parameter approaches the control value, then treatment is considered efficacious. In other embodiments, the individuals in the control population have not received prior treatment and have been diagnosed with Friedreich's ataxia. In such cases, if the value of the measurable biomarker or clinical parameter approaches the control value, then treatment is considered inefficacious.

In other methods, a subject who is not presently receiving treatment but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation) determined in population of subjects after undergoing a course of treatment. Alternatively, the measured value in the subject can be compared with a control value in populations of prophylactically treated subjects who remain free of symptoms of disease, or populations of therapeutically treated subjects who show amelioration of disease characteristics. In such cases, if the value of the measurable biomarker or clinical parameter approaches the control value, then treatment is considered efficacious and need not be resumed. In all of these cases, a significant difference relative to the control level (i.e., more than a standard deviation) is an indicator that treatment should be resumed in the subject.

9. Screening for Agents

Assays to identify compounds useful for preventing, reducing, delaying or inhibiting symptoms of Friedreich's ataxia can be performed in vitro. As demonstrated herein, candidate agents can be contacted with a population of test cells in the presence of a lethal or sub-lethal concentration of an inhibitor of the thioredoxin reductase pathway, wherein an agent that prevents, reduces, delays or inhibits one or more symptoms of Friedreich's ataxia increases cell viability and/or prevents cell death in the presence of the inhibitor of the thioredoxin reductase pathway. The increase in cell viability and/or prevention of cell death can be determined in comparison to a control population of cells that have not been contacted with the candidate agent. Cell viability in a populations of cells can be determined using any known method.

In some embodiments, the inhibitor of the thioredoxin reductase pathway is selected from the group consisting of antimycin A, auranofin, buthionine sulfoximine (BSO), carmustine, diamide, diethyl maleate, ethanol, hydrogen peroxide, L glutathione, phenethyl isothiocyanate (PEITC), dichloronitrobenzene, N-methyl-2-pyrrolidinone, and mixtures and analogs thereof. In some embodiments, the inhibitor of the thioredoxin reductase pathway is selected from the group consisting of auranofin, diamide, and mixtures and analogs thereof.

In various embodiments, agents of interest can be further selected for their ability to induce and/or increase the expression levels of frataxin, measured at the protein or mRNA level. Expression levels of frataxin can be determined in cells or animals models, such as described in the present examples. In some embodiments, agents of interest are selected that increase viability and/or prevent cell death by at least about 1.4-fold, for example, at least about 1.5-fold, 1.6-fold, 1.7-fold, 1.8-fold, 1.9-fold, 2.0-fold, or more, in comparison to a control population of cells that have not been contacted with the candidate agent. In some embodiments, agents of interest are selected that increase viability and/or prevent cell death with a low EC50 concentration, for example, an EC50 concentration of less than about 5 μM, for example, less than about 4 μM, 3 μM, 2 μM, 1 μM, 0.5 μM or less. Active agents of interest can be further confirmed by testing their ability to increase viability and/or prevent cell death in a dose-dependent manner.

In some embodiments, the candidate agent is a small organic compound, a polypeptide, an antibody or fragment thereof, an amino acid or analog thereof, a carbohydrate, a saccharide or disaccharide, or a polynucleotide.

In some embodiments, the population of cells is a population of fibroblast cells. In some embodiments, the population of cells is a population of neuronal or nerve cells. In some embodiments, the population of cells is a population of dorsal root ganglion cells.

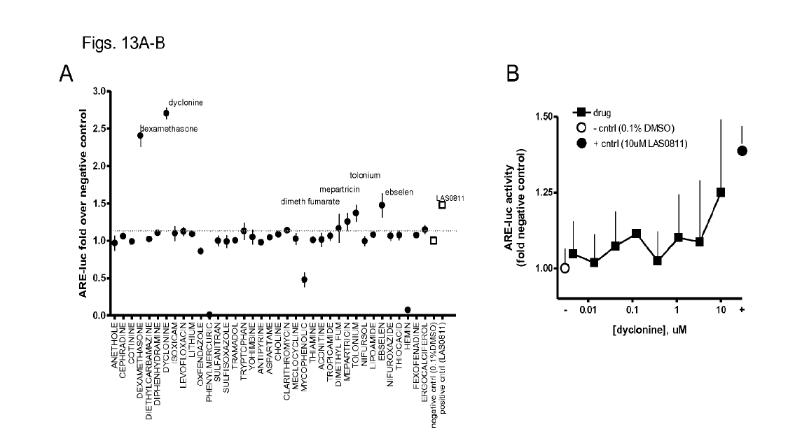

The invention provides further screening methods in which agents are initially screened to determine whether they have an agonist effect on the thioredoxin reductase and NRF2 pathway. Agents having such an effect can then be screened in a cellular or animal model of Friedreich's ataxia to determine whether an agent has an activity providing an indication of utility in treatment of Friedreich's ataxia. Commercial kits for determining agonism of the NRF2 pathway are available (e.g., PathHunter® U2OS Keap1-NRF2 Functional Assay from DiscoveRx) and an example of such an assay is provided in the Examples (FIG. 13 and description). The secondary screen can be performed in cellular or animal models of Friedreich's ataxia, for example, cells from subjects with Friedreich's ataxia or transgenic animal models thereof. One such model, is a mouse with a homozygous knocked out endogenous frataxin gene and a transgene encoding a human frataxin protein, the transgene including a triplet repeat conferring Friedreich's ataxia susceptibility. The activity measured in the secondary screen can be an increased in frataxin levels, which in a transgenic animal can be measured in spleen, liver or brain as illustrated by the present examples. Alternatively, the activity measured can be an improvement or at least reduced rate of decline of neurological and motor function.

The screening methods of the invention can be conveniently carried out using high-throughput methods. In some embodiments, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known. Such combinatorial chemical libraries include, but are not limited to, peptide libraries (see, e.g. U.S. Pat. No. 5,010,175, Furka, Int J Pept Prot Res 37:487-493 (1991) and Houghton, et al., Nature 354:84-88 (1991)). Other chemistries for generating chemical diversity libraries can also be used. Such chemistries include, but are not limited to peptoids (e.g., WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (Hobbs, et al, Proc Nat Acad Sci USA 90:6909-6913 (1993)), vinylogous polypeptides (Hagihara, et al., J Amer Chem Soc 114:6568 (1992)), nonpeptidal peptidomimetics with glucose scaffolding (Hirschmann, et al., J Amer Chem Soc 114:9217-9218 (1992)), analogous organic syntheses of small compound libraries (Chen, et al., J Amer Chem Soc 116:2661 (1994)), oligocarbamates (Cho, et al., Science 261:1303 (1993)) and/or peptidyl phosphonates (Campbell, et al., J Org Chem 59:658 (1994)), nucleic acid libraries, peptide nucleic acid libraries (see, e.g. U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang, et al., Science 274:1520-1522 (1996) and U.S. Pat. No. 5,593,853), small organic molecule libraries (see, e.g., benzodiazepines, Baum, C&EN, January 18, page 33 (1993), isoprenoids, U.S. Pat. No. 5,569,588), thiazolidinones and metathiazanones, U.S. Pat. No. 5,549,974 pyrrolidines, U.S. Pat. Nos. 5,525,735 and 5,519,134, morpholino compounds, U.S. Pat. No. 5,506,337 benzodiazepines, U.S. Pat. No. 5,288,514, and the like).

Devices for the preparation of combinatorial libraries are commercially available (see, e.g., 357 MPS, 390 MPS, Advanced Chem Tech. Louisville Ky.; Symphony, Rainin, Woburn, Mass.; 433A Applied Biosystems, Foster City, Calif.; 9050 Plus, Millepore, Bedford. MA). In addition, numerous combinatorial libraries are themselves commercially available (see, e.g., ComGenex, Princeton, N.J.; Tripos, Inc, St Louis, Mo.; 3D Pharmaceuticals, Eaton, Pa.; Martek Biosciences, Columbia, Md.). Libraries of FDA approved compounds are commercially available and find use (e.g., from Enzo Life Sciences (enzolifesciences.com); and Microsource Discovery Systems (msdiscovery.com)). Chemical libraries with candidate agents selected for bioavailability and blood-brain barrier penetration also find use, and are commercially available, e.g., from ChemBridge (chembridge.com) and Prestwick Chemical (prestwickchemical.fr). Further libraries of chemical agents that find use are available, e.g., from Evotec (evotec.com); Magellan BioScience Group (magellanbioscience.com); and Cellumen (cellumen.com).

In high throughput assays of the invention, it is possible to screen up to several thousand different candidate agents in a single day. In particular, each well of a microtiter plate can be used to run a separate assay against a selected potential candidate agent, or, if concentration or incubation time effects are to be observed, every 5-10 wells can test a single modulator. Thus, a single standard microtiter plate can assay about 100 (e.g., 96) candidate agents. Multiwell plates with greater numbers of wells find use, e.g., 192, 384, 768 or 1536 wells. If 1536-well plates are used, then a single plate can easily assay from about 100 to about 1500 different compounds. It is possible to assay several different plates per day. Assay screens for up to about 6,000-20,000 different compounds are possible using the integrated systems of the invention.

### EXAMPLES

The following examples are offered to illustrate, but not to limit the claimed invention.

### Example 1

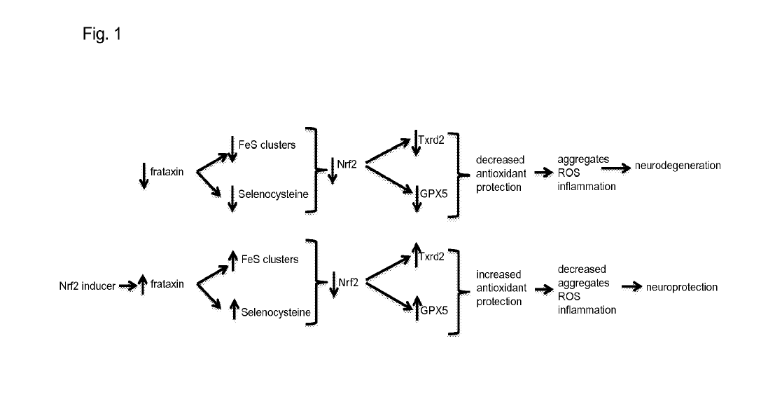

Mechanism of pathophysiology of Friedreich's ataxia and rescue with biochemical agents. FIG. 1 (top panel) illustrates a pathophysiological model for Friedreich's ataxia based on dorsal root ganglion microarrays and biochemical investigation and drug screening. Frataxin is involved in mitochondrial iron-sulfur cluster biogenesis, and facilitates mitochondrial selenocysteine metabolism, which is essential to the protection of mitochondria from oxidative stress, which is primarily mediated by the selenoenzymes Thioredoxin reductase (Txrd2), and glutathione peroxidase (GPX5). As a result of deficiency of frataxin, these selenoenzymes have decreased activity, and Nrf2 declines, the result is decreased mitochondrial antioxidant protection, increased aggregates, reactive oxygen species, inflammation and neurodegeneration. In addition frataxin interacts with NFS1 of the 2Fe2S cluster biogenesis machinery, necessary for glutaredoxin 2 and ferredoxin 2 function. Reduced function of glutaredoxin 2 and fenedoxin 2 leads to deficiencies of thioredoxin reductase, decreased mitochondrial antioxidant protection, increased aggregates, reactive oxygen species, inflammation and neurodegeneration. In FIG. 1 (bottom panel) we observe that inducers of Nrf2 increase frataxin expression, increase selenocysteine metabolism and Txrd2 and GPX5 activity, and increase iron-sulfur cluster biogenesis, and promote cellular protection.

### Example 2

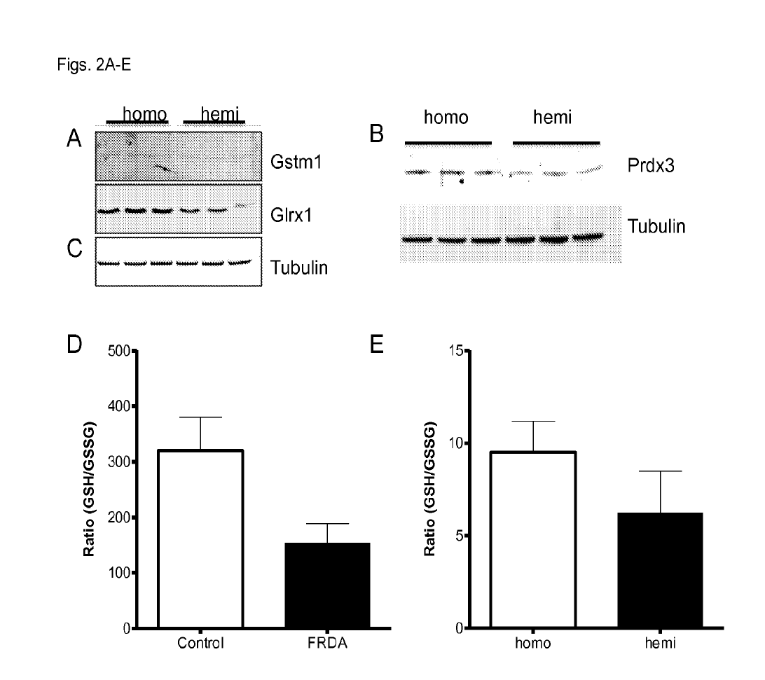

Multiple proteins directly or indirectly reduced by thioredoxin reductase are deficient in YG8 mice. DRGs of YG8 mice were microdissected and protein expression of genes measured. Peroxiredoxin-3, Glutaredoxin-1 and Glutathione-S-transferase-1 were each decreased (FIG. 2A-C). Glutathione is the most important redox buffer in the cell and low GSH/GSSG indicates increased oxidative stress. It has been shown that FRDA patient lymphoblasts had decreased GSH/GSSG as a consequence of elevated GSSG levels (Tan, et al., Hum Mol Genet (2003) 12:1699-1711) (FIG. 2D). Similarly, hemizygous YG8 mice cerebellum and DRG tissue had significantly more and about twice the level of GSSG than homozygous mice (0.23 vs. 0.14 micromol/g), causing a decreased GSH/GSSG ratio, demonstrating increased oxidative stress in this tissue (FIG. 2E).

### Example 3

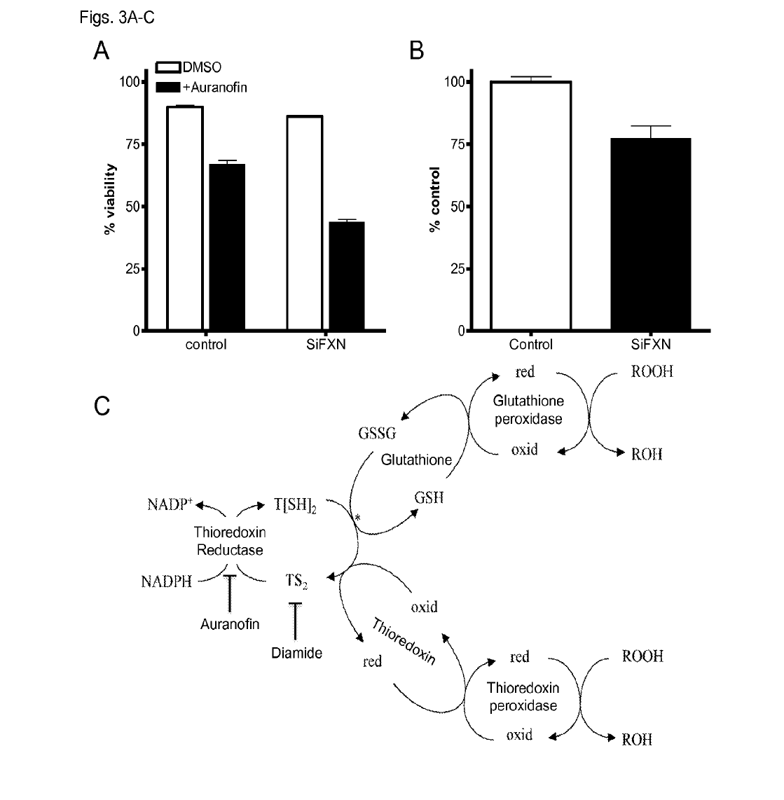

Frataxin deficiency causes thioredoxin reductase deficiency, and decreased antioxidant activity and expression. A connection was sought between the multiple thiol-related antioxidants (peroxiredoxins, glutaredoxins, thioredoxins, GSSG) that were decreased in microarray and Westerns of the YG8 DRGs; most of them are reduced by thioredoxin reductase (FIG. 3C). Thioredoxin reductase, in addition to reducing the 2Fe2S-cluster containing glutaredoxin 2, also reduces peroxiredoxins, thioredoxins, and glutathione, which are used as a mitochondrial antioxidant system. Frataxin was knocked down using siRNA in HeLa cells and decreased thioredoxin reductase activity was observed (FIG. 3A). Frataxin deficiency and thioredoxin reductase deficiency additively caused cell death (FIG. 3B).

### Example 4

Overall, a novel FRDA screening assay based on the thioredoxin reductase pathway identified dyclonine and other drugs that protected FRDA cells from diamide induced oxidative stress. Friedreich's ataxia is an inherited mitochondrial neurodegenerative disease that results from a deficiency in frataxin, a neuroprotective mitochondrial protein. We demonstrated that neurons and patient cells with a defect in frataxin died when exposed to the thioredoxin reductase oxidants diamide and auranofin. We screened a library of 1600 drugs for their ability to rescue this degeneration, and identified multiple neuroprotective compounds, including dyclonine, dimethyl fumarate, methylene blue, and nifursol.

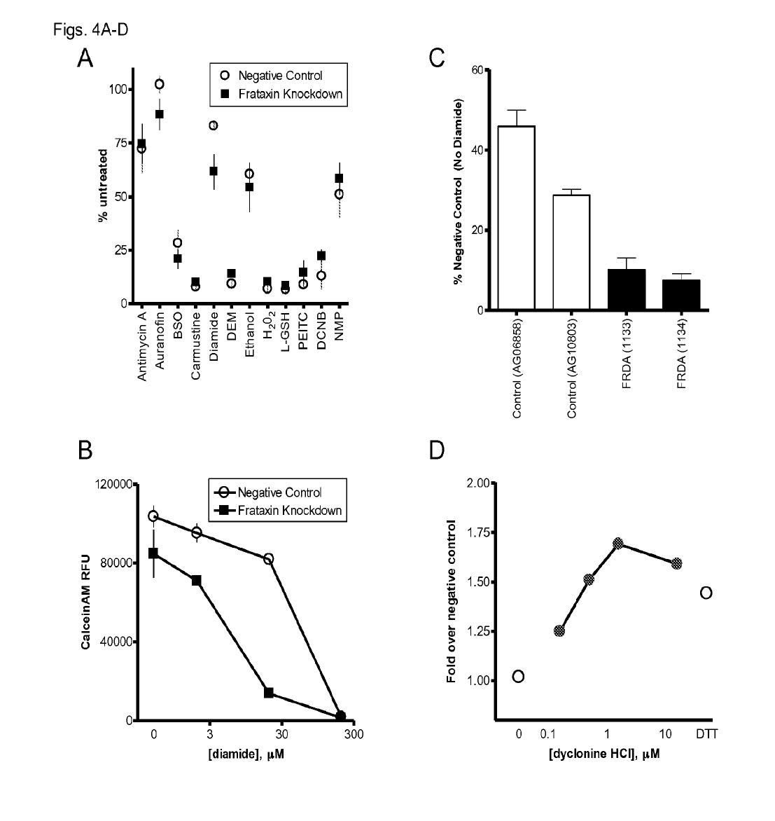

Microarray of dorsal root ganglion neurons from the YG8 mouse model of Friedreich's Ataxia suggested there was a deficiency in multiple thiol-related antioxidants. Therefore, 11 inhibitors of these antioxidants were tested in siRNA-mediated frataxin deficient 50B11 dorsal root ganglion cell line. The results demonstrated that neurons with frataxin deficiency died more rapidly when treated with the thioredoxin oxidant diamide, and the thioredoxin reductase inhibitor auranofin (FIG. 4A). This sensitivity was dose-dependent in DRG neurons (FIG. 4B) and was confirmed in Friedreich's patient fibroblasts (FIG. 4C), and could be reversed by the reductant DTT (FIG. 4D). The major mitochondrial antioxidant system is thioredoxin reductase. Auranofin is a specific inhibitor of thioredoxin reductase, and diamide is a known oxidizer of thioredoxin. Thus the diamide screen identifies compounds that rescue from thioredoxin reductase deficiency, which include inducers of Nrf2, which are known to induce thioredoxin reductase and other antioxidant functions.

The cell-based assay was further optimized for high-throughput screening in 96-well plates, with an excellent screening window and low variability, represented by a Z′ value of 0.75 (n=5) and was used to screen a library of 1600 drugs that have been approved for clinical use in the USA. Drugs that rescued at DMSO mean+two standard deviations were repeated an additional two times. Compounds that rescued in >2 screens advanced to secondary screening, which included replication of protective effect in a concentration-dependent manner, 0.01-10 μM. An example of dose dependent protection by dyclonine (FIG. 4D) The three most prominent functional groups were inhibitors of the arachidonic acid pathway, and sulfur-containing compounds with known effects on mitochondria, antioxidants, and Nrf2 inducers. Additional mechanisms are listed below. Of the 40 neuroprotective drugs identified, 20 increased frataxin in FRDA patient cells, and four (dyclonine, dimethyl fumarate, methylene blue and nifursol) increased frataxin in brains and other tissues of the animal model of FRDA.

Mechanism of Action of Drugs.

Protective drugs isolated in the diamide screen can work by multiple mechanisms of action. These include without limitation:

1) the induction of frataxin, which has neuroprotective effects;
2) the induction of Nrf2, which has neuroprotective effects;
3) the inhibition of thioredoxin reductase, which is known to induce Nrf2, which has protective effects;
4) the Nrf2-dependent induction of frataxin; and
5) the increase in histone methylysine transferase, which increases the expression of multiple neuroprotective genes including frataxin;
6) the ferredoxin-dependent induction of frataxin.
7) Also, the potentiation of mitochondrial function (lipoic/thioctic acid are supplements of mitochondrial function).
8) As direct antioxidants, e.g., ebselen.
9) As inducers of mitochondrial iron-sulfur cluster biogenesis.
10) As inducers of PGC-1a, which induces mitochondrial functions and is neuroprotective.

Diamide Screening Method Format: On the day one of the assay, cell density was determined using the Vi-Cell counter, and a volume corresponding to 5,500 human FRDA patient fibroblasts per well was aliquoted into 96-well black/clear poly-d-lysine coated plates in growth media, and the cells were allowed 3 hours to attach. Drugs (10 mM stock in DMSO) are dispensed into wells after an intermediate dilution in PBS, giving a final DMSO concentration of 0.1% using an electronic multichannel pipette. Test compounds were tested at 10 micromolar. There were 8 negative control (0.1% DMSO only) and 8 positive control (300 micromolar DTT) wells on each plate. Plates were incubated at 37 C and 5% CO2 for twenty-four hours after which 200 micromolar diamide was added to all wells. Plates were incubated for an additional 16 hours as before. Cells were washed with PBS and incubated with Calcein-AM cell viability dye (Invitrogen, Carlsbad, Calif., USA) and fluorescence was read with a PolarStar Omega plate reader (BMG LabTech, Cary, N.C.). Hits were scored as Basal Median+3XMAD.

### Example 5

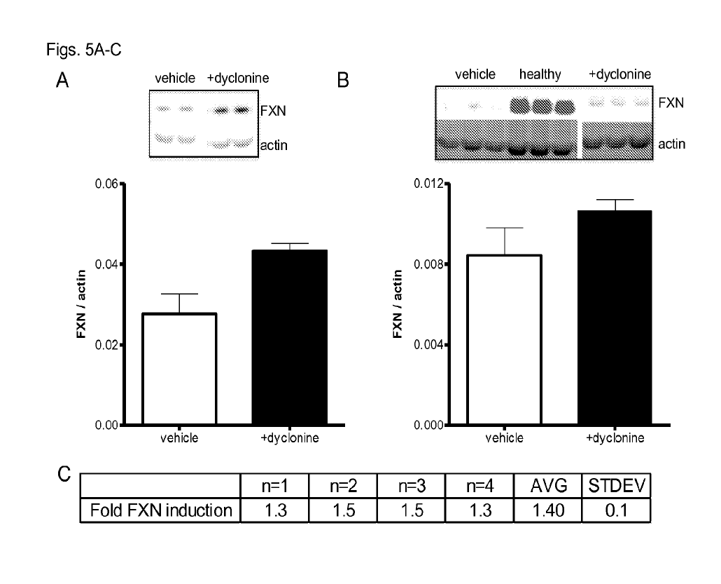

Dyclonine induces frataxin expression in FRDA lymphoblasts and HeLa cells. To test if one mechanism of protection from diamide toxicity for dyclonine was an increase in frataxin protein levels, HeLa cells or FRDA patient lymphoblasts were cultured for 48 hr in the presence of 10 micromolar dyclonine in 6 well dishes. Cells were harvested and whole cell lysates were analyzed by Western blot (protocol below). Representative blots are shown for HeLa cells (FIG. 5A) and FRDA and healthy siblings lymphoblasts (FIG. 5B), as well as densitometry normalization to actin. Dyclonine induction of FXN levels was consistent over multiple experiments (FIG. 5C).

Western Blot Protocol: Forty micrograms of whole-cell lysates were analyzed on 4-10% Bis-Tris gels (Invitrogen, Carlsbad, Calif., USA). Electrophoresis was carried out according to the manufacturer's instruction. After electrophoresis, the proteins were transferred to nitrocellulose membranes by iBlot device (Invitrogen). The membranes were blocked with blocking buffer (Odyssey, Lincoln, Nebr., USA) for 1 h and incubated overnight with primary antibodies in blocking buffer: anti-frataxin anti-β-actin, tubulin, (Sigma). Afterwards, the membranes were incubated with a corresponding pair of IRDye 680CW- and IRDye 800CW-coupled (Odyssey) secondary antibodies for 1 h. The membranes were washed four times with 1× Tris-buffered saline with Tween 20 and proteins were visualized with a LI-COR infrared imager (Odyssey). The pictures were processed by Odyssey version 3.0 infrared imaging software.

### Example 6

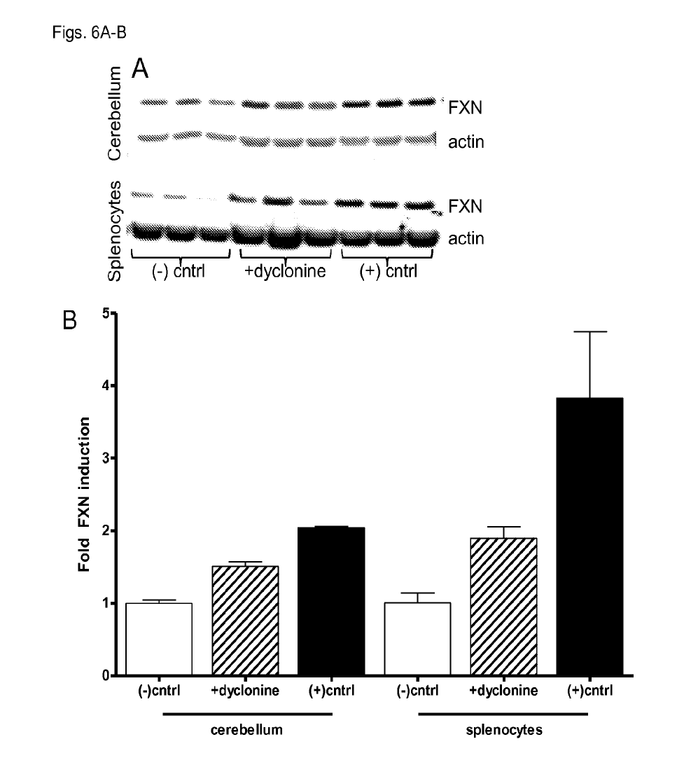

Dyclonine increases frataxin levels in vivo. To determine ability of dyclonine to reverse the in vivo FXN protein defect, the YG8 FRDA transgenic mouse model was chosen. Hemizygous animals with the least frataxin were separated into vehicle and treatment groups. Homozygous mice (two copies of FXN gene) were used as positive control. Animals were dosed daily with 1 mg/kg dyclonine via intraperitoneal injection for 6 days. At the end of the study, the animals were sacrificed, and processed for biochemical analysis, i.e. Western blots of treated and vehicle groups of Cerebellum and splenocyte frataxin level. Western blot of cerebellum and splenocytes is shown (FIG. 6A) and densitometry of FXN/actin normalization (FIG. 6B) showing dyclonine induces FXN expression in vivo by 1.5-2 fold.

### Example 7

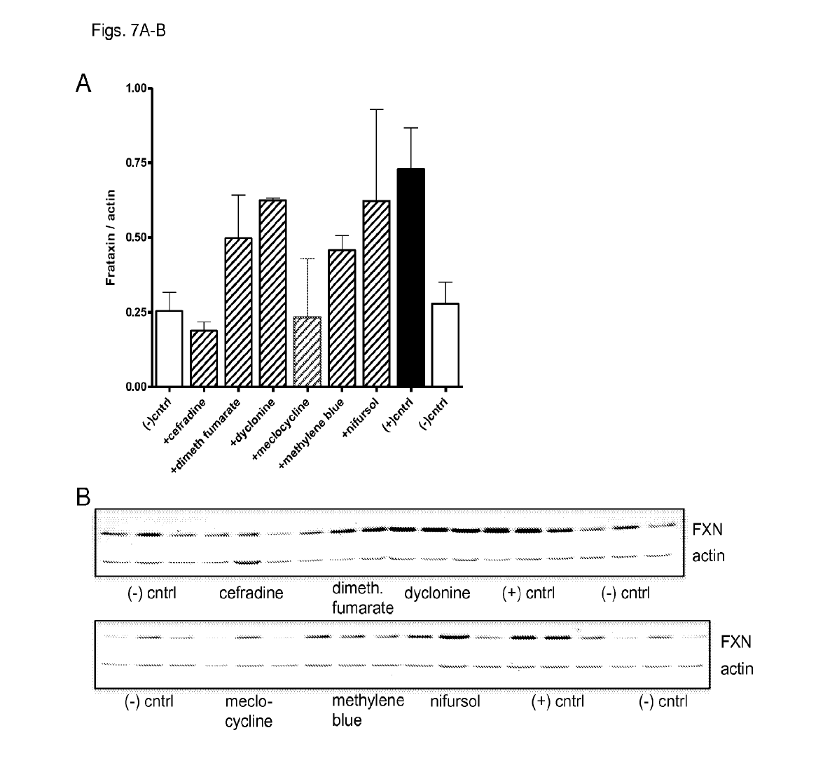

Drugs in addition to dyclonine increase FXN levels in vivo. 20 of the original 40 neuroprotective drugs were shown to increase FXN levels in FRDA patient cells. Of these 20, 8 were tested in the YG8 transgenic mouse model. In addition to dyclonine, dimethyl fumarate, methylene blue, and nifursol were observed to increase frataxin in brain (7A,B) Animals were dosed daily with 1-10 mg/kg drug via intraperitoneal injection for 6 days. At the end of the study, the animals were sacrificed, and processed for biochemical analysis, i.e. Western blots (protocol above) of treated and vehicle groups of Cerebellum and splenocyte frataxin level. Western blot of cerebellum is shown (FIG. 7B) and densitometry of FXN/actin normalization (FIG. 7A) showing dimethyl fumarate, dyclonine, methylene blue, and nifursol induce FXN expression in vivo by 1.5-2 fold.

### Example 8

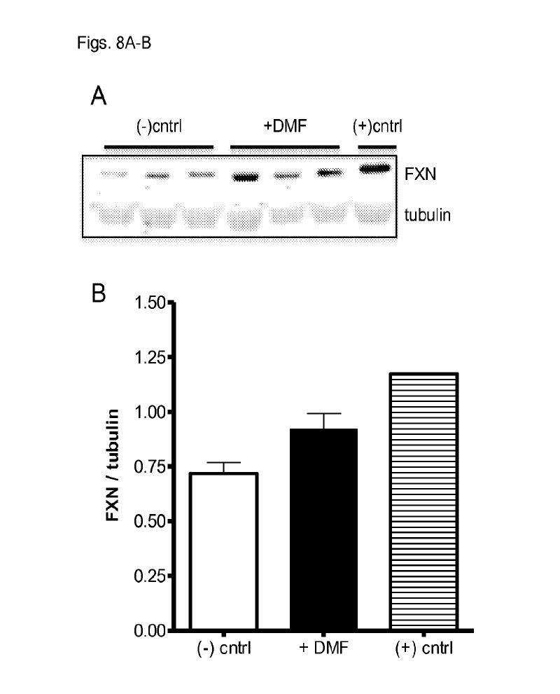

Additional in vivo tests also revealed dimethyl fumarate as FXN inducer in mouse model. To determine ability of dimethyl fumarate to reverse the in vivo FXN protein defect, the YG8 FRDA transgenic mouse model was chosen. Hemizygous animals with largest FXN defect, were separated into vehicle and treatment groups. Homozygous mice were used as positive control Animals were dosed daily with 5 mg/kg dimethyl fumarate via intraperitoneal injection for 6 days. A non-specific HDAC inhibitor was used as a positive control, dosed at 1 mg/kg. At the end of the study, the animals were sacrificed, and processed for biochemical analysis, i.e. Western Blots of treated and vehicle groups of cerebellum and splenocyte frataxin level. Western blot of cerebellum is shown (FIG. 8A) and densitometry of FXN/actin normalization (FIG. 8B) showing dimethyl fumarate induces FXN expression significantly (p<0.05) in vivo by 1.5-2 fold.

### Example 9

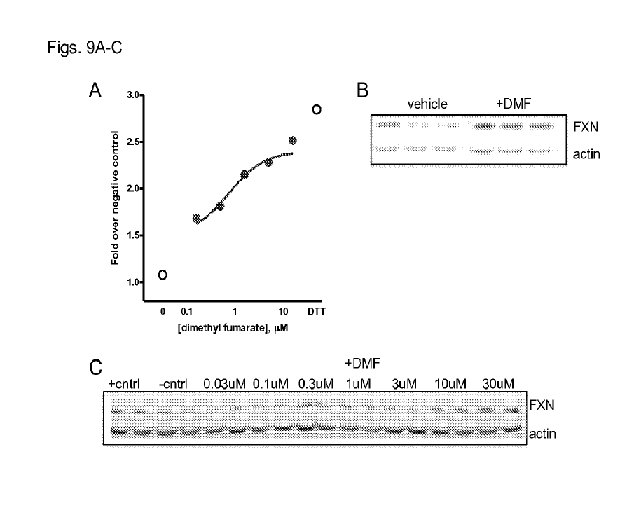

Dimethyl fumarate protects from diamide and induces frataxin accumulation in FRDA cells. An additional hit in the diamide screening assay in FRDA fibroblasts was dimethyl fumarate (protocol above). This protection from diamide toxicity was dose-dependent (FIG. 9A), and produced maximum effects of 1.9±0.2 fold increases from baseline with an EC50=1.3±0.8 μM (n=3). To test if the mechanism of protection from diamide toxicity was an increase in frataxin protein levels, HeLa cells or FRDA patient lymphoblasts were cultured for 48 hr in the presence of 0.03-30 micromolar dimethyl fumarate in 12-well dishes. Cells were harvested and whole cell lysates were analyzed by Western blot (protocol above). Representative blots are shown for HeLa cells (FIG. 9A) and FRDA lymphoblasts (FIG. 9B). FXN was induced 1.37 fold±0.1 (n=3) in HeLa cells, and 2.4 fold±0.9 (n=3) in FRDA lymphoblasts. Significant FXN induction was observed at 1, 10 and 30 micromolar dimethyl fumarate (FIG. 9C).

### Example 10

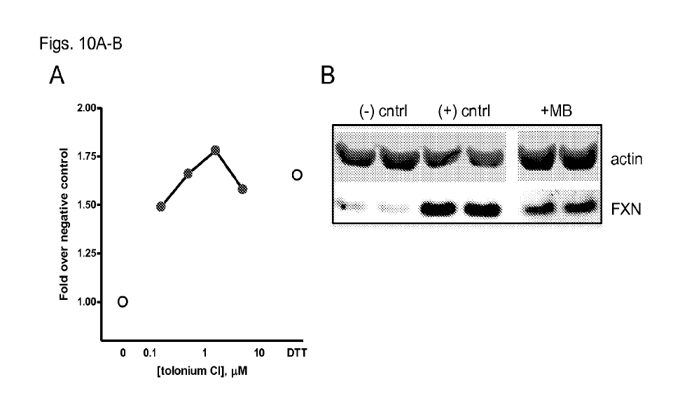

Phenathiazines protect from diamide and induce frataxin accumulation in FRDA cells. An additional hit in the diamide screening assay (protocol above) in FRDA fibroblasts was the phenathiazine, tolonium cl. This protection from diamide toxicity was dose dependent (FIG. 10A), and produced maximum effects of 1.9±0.1 fold increases from baseline with an EC50=0.1±0.01 μM (n=3). To test if the mechanism of protection from diamide toxicity was an increase in frataxin protein levels, the phenothiazines tolonium cl and methylene blue were tested. FRDA patient lymphoblasts were cultured for 48 hr in the presence of 10 nanomolar methylene blue in 12 well dishes. Cells were harvested and whole cell lysates were analyzed by Western blot (protocol above). Representative blots are shown for FRDA lymphoblasts (FIG. 10B). Fold FXN induction in FRDA lymphoblasts was 1.7 fold±0.1 (n=2).

### Example 11

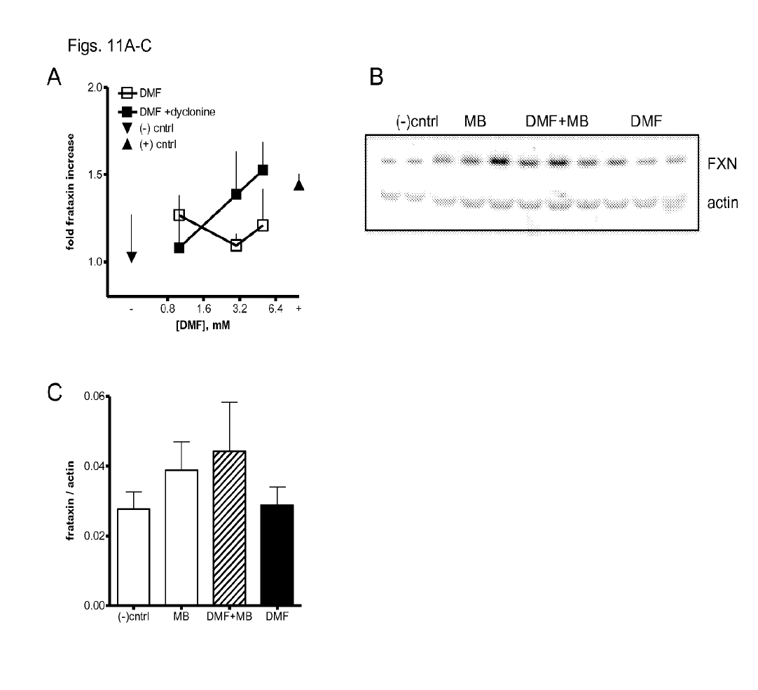

Synergy of dyclonine, methylene blue and dimethyl fumarate to induce frataxin expression. To evaluate potential synergy of identified FXN-inducing drugs, a dose-response of dimethyl fumarate in the absence or presence of 5 micromolar dyclonine was determined in FRDA lymphoblasts incubated for 48 hours in 96 well plates. Whole cells were fixed and permeabilized and for analyzed using in-cell Western technique (protocol below). Dyclonine potentiated DMF FXN induction in FRDA lymphoblasts (FIG. 11A).

To test if Methylene blue also potentiates DMF-mediated FXN induction, FRDA patient lymphoblasts were cultured for 48 hr in the presence of 3 micromolar dimethyl fumarate and 3 micromolar methylene blue in 6 well dishes. Cells were harvested and whole cell lysates were analyzed by Western blot (protocol above). Representative blots are shown for HeLa cells (FIG. 11B-C), showing Methylene blue also potentiates DMF FXN induction in vitro.

In-Cell Western Blot Protocol: 50,000 cells/well were plated in black walled clear bottom 96-well poly-d-lysine coated plates. After drug treatment, media was removed by aspiration and cells were fixed by addition of 100 ul/well of 3.7% formaldehyde in PBS. After 30 minutes, fixing solution was removed and 100 ul/well of 0.1% TritonX100 in PBS was added to all wells. After 40 minutes, cells were blocked with 100 ul blocking buffer (Odyssey, Lincoln, Nebr., USA) for 1 h and incubated with primary antibodies in blocking buffer: anti-frataxin (Mitosciences, Eugene, Oreg.) anti-β-actin (Sigma) for one hour. Cells were washed three times with 1× Tris-buffered saline with Tween 20, and then incubated with 100 ul/well of IRDye 680CW- and IRDye 800CW-coupled (Odyssey) secondary antibodies for 1 h. The cells were washed three times with 1× Tris-buffered saline with Tween 20 and signal was visualized with a L1-COR infrared imager (Odyssey). The pictures were processed by Odyssey version 3.0 infrared imaging software.

### Example 12

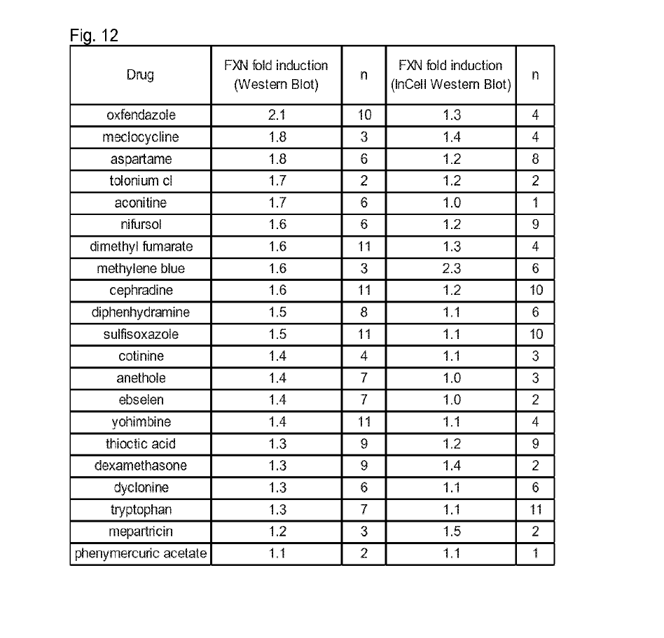

Of the original 40 drugs identified as protective in diamide screening assay, 20 were found reproducibly to increase FXN protein levels using traditional western blot or in cell western blot methods, showing fold increase of FXN protein expression in human cells; n=number of experiments (FIG. 12).

### Example 13

To evaluate the mechanism of FXN induction of the 40 drugs that protected from diamide, the potency of each to activate the Nrf2 target antioxidant response element (ARE) was evaluated. 10 of the 20 frataxin inducers interacted with the Nrf2 pathway in a significant way, either positively or negatively. The dashed line=mean+2×SD. Drugs that induced Nrf2/ARE activity at mean+2× Standard deviation of the vehicle control included dyclonine, dexamethasone, mepartricin, dimethyl fumarate, tolonium cl, and ebselen (FIG. 13A). Dyclonine's induction of ARE/Nrf2 activity was dose-dependent (FIG. 13B).

Nrf2/ARE Luciferase Assay.

The Nrf2 reporter HeLa cell line was generated using the Lenti Antioxidant Response Reporter (Qiagen, Valencia, Calif.) system, which expressed firefly luciferase gene and the ARE transcriptional response element. 15,000 cells/well were plated in 90 ul in phenol-free DMEM media in white wall/bottom 96 well plates. Drugs were added and plates incubated at 37 C for 24 hours. 75 microliters of Bright Glo Luciferase Assay Reagent (Promega, Madison, Wis.) with combined lysis solution was added to all wells and allowed to incubate for 5 minutes at room temperature. Luminescence was then read with a PolarStar Omega plate reader (BMG LabTech, Cary, N.C.).

### Example 14

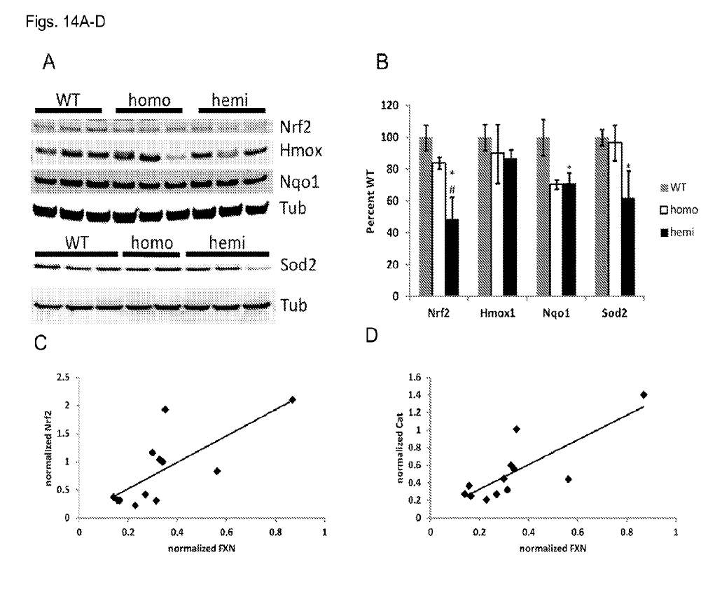

We determined whether the Nrf2 protein was deficient in target dorsal root ganglion (DRG) tissue in the available YG8 model of Friedreich's ataxia. DRG tissue was dissected from wild-type, homozygous and hemizygous (affected) mice, protein extracted, and electrophoresed, blotted and quantified. There was a clear deficiency of protective Nrf2 protein in hemizygotes (FIGS. 14 A, B, and C), and the transcriptional targets of Nrf2, i.e. Nqo1 and SOD2, were also decreased (FIGS. 14 B, and C), frataxin expression was significantly correlated with Nrf2 expression (FIG. 14D), frataxin expression was significantly correlated with the Nrf2 target catalase expression.

In addition to neuroprotection, these compounds increased expression of the mitochondrial protein frataxin in patient cells and the animal model of the disease. Their activity is synergistic with respect to the induction of frataxin. We showed that these compounds induced the Nrf2 pathway. Thus we describe three neuroprotective drugs that induce expression of the neuroprotective protein frataxin and the neuroprotective protein Nrf2.

The examples and embodiments described herein are for illustrative purposes only and that various modifications or changes in light thereof are to be included within the spirit and purview of this application and scope of the appended claims. Each embodiment, aspect, element, feature, step or the like can be used in combination with any other unless the context requires otherwise. For example, although the invention is sometimes described with reference to Friedreich's ataxia, the disclosure also apply to other neurodegenerative diseases mentioned. All publications (including accession numbers, websites and the like), patents, and patent applications cited herein are hereby incorporated by reference in their entirety for all purposes to the same extent as if so individually denoted. To the extend a reference, such as an accession number is associated with different content at different times, the version in effect at the effective filing date of the application is meant. Effective filing date means the actual filing date or earlier filing date in which such reference was cited.

1. A method for reducing, delaying or inhibiting Friedreich's ataxia in a subject in need thereof comprising administering to the subject an effective amount of
a compound conforming to formula (II): 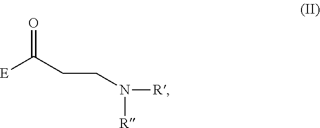
or a pharmaceutically acceptable salt thereof;
wherein E is substituted or unsubstituted aryl, or substituted or unsubstituted heteroaryl; and R′ and R″ taken together with the N to which each is bound form a primary, secondary or tertiary amine, or together with the N to which each is bound, R′ and R″ form a cyclic amine group.

2. The method of claim 1, wherein E is an R³-substituted C₍₆₋₁₀₎ aryl, or R³-substituted C₍₂₋₉₎ heteroaryl, R³ can be a substituent on any available position of the aryl or heteroaryl ring, R³ is hydrogen, halogen (F, Cl, Br, I), —CN, —OH, —NH₂, —COOH, —CF₃, (C₍₂₋₈₎ alkoxy), R⁴-substituted C₍₁₋₈₎ alkyl, unsubstituted C₍₁₋₈₎ alkyl, R⁴-substituted C₍₁₋₈₎ heteroalkyl, unsubstituted C₍₁₋₈₎ heteroalkyl, R⁴-substituted C₍₃₋₇₎ cycloalkyl, unsubstituted C₍₃₋₇₎ cycloalkyl, R⁴-substituted C₍₂₋₇₎ heterocycloalkyl, unsubstituted C₍₂₋₇₎ heterocycloalkyl, R⁴-substituted C₍₆₋₁₀₎ aryl, unsubstituted C₍₆₋₁₀₎ aryl, R⁴-substituted C₍₂₋₉₎ heteroaryl or unsubstituted C₍₂₋₉₎ heteroaryl, or a pharmaceutically acceptable salt thereof, wherein R⁴ is in each instance selected from the group consisting of halogen (F, Cl, Br, I), —CN, —OH, —NH₂, —COOH, —CF₃, —OCH₃, —OC₂H₅, —OC₃H₇, and —OCOCH₃.

3. The method of claim 1, wherein R′ and R″ taken together with the N to which each is bound can be a 3-membered cyclic aziridines, 4-membered cyclic azetidines, 5-membered cyclic pyrrolidines, 6-membered cyclic piperidines, 7-membered cyclic azepanes, 8-membered cyclic azocanes.

4. The method of claim 1, wherein the compound conforms to formula (IIA): 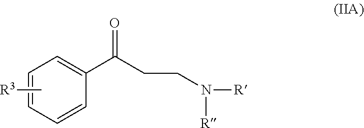
or a pharmaceutically acceptable salt thereof;
wherein R³ is hydrogen, halogen (F, Cl, Br, I), —CN, —OH, —NH₂, —COOH, —CF₃, —OCH3, —OC2H5, —OC3H7, —OCOCH3, —OR⁴, R⁴-substituted or unsubstituted alkyl (e.g., alkyl of 2 to 8 carbon atoms), R⁴-substituted or unsubstituted heteroalkyl, R⁴-substituted or unsubstituted cycloalkyl, R⁴-substituted or unsubstituted heterocycloalkyl, R⁴-substituted or unsubstituted aryl, or R⁴-substituted or unsubstituted heteroaryl, or a pharmaceutically acceptable salt thereof.

5. The method of claim 4, wherein the compound conforms to formula (IIB): 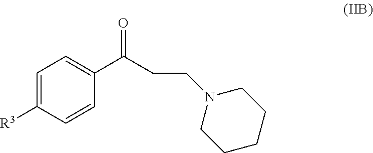
or a pharmaceutically acceptable salt thereof.

6. The method of claim 1, wherein the compound is dyclonine or a pharmaceutically acceptable salt thereof.

7. The method of claim 1, wherein the subject is co-administered an effective amount of DMF or methylene blue or a pharmaceutically acceptable salt thereof.

8. The method of claim 6, wherein the dyclonine is administered in a dose of a least 1 mg/kg.

9. The method of claim 6, wherein the dyclonine is administered in a dose of 1-500 mg subject, preferably at least 100 mg/subject.

10. The method of claim 6, wherein the dyclonine is formulated as a controlled-release composition.

11. The method of claim 6, wherein the dyclonine is administered intramuscularly, intravenously, subcutaneously or orally.

12. The method of claim 1, wherein the subject is free of other known diseases amenable to treatment with dyclonine.

13. The method of claim 1, wherein the subject is monitored for an increase in level of frataxin responsive to the administering.

14. A controlled-release formulation of dyclonine or a single-use formulation of dyclonine containing at least 100 mg dyclonine.

15. (canceled)

16. The method of claim 1, wherein the dyclonine is in the form of a pharmaceutically acceptable salt other than HCl.

17. A method for reducing, delaying or inhibiting Friedreich's ataxia in a subject in need thereof comprising administering to the subject an effective amount of
a compound of formula (I): 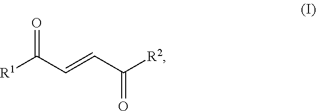
or a pharmaceutically acceptable salt thereof;
wherein R¹ and R² are independently selected from —CH₃₋ₙEₙ, OH, O⁻, and (C₁₋₈) alkoxy (branched or unbranched), provided that at least one of R¹ and R² is (C₁₋₈) alkoxy, E is an electron withdrawing group.

18. The method of claim 17, wherein E is selected from the group consisting of —NO₂, —N(R₂), —N(R₃)⁺, —N(H₃)⁺, —SO₃H, —SO₃R′, —S(O₂)R′ (sulfone), —S(O)R′ (sulfoxide), —S(O₂)NH₂ (sulfonamide), —SO₂NHR′, —SO₂NR′₂, —PO(OR′)₂, —PO₃H₂, —PO(NR′₂)₂, pyridinyl (2-, 3-, 4-), pyrazolyl, indazolyl, imidazolyl, thiazolyl, benzothiazolyl, oxazolyl, benzimidazolyl, benzoxazolyl, isoxazolyl, benzisoxazolyl, triazolyl, benzotriazolyl, quinolinyl, isoquinolinyl, quinazolinyl, pyrimidinyl, a 5 or 6-membered heteroaryl with a C—N double bond optionally fused to a 5 or 6 membered heteroaryl, pyridinyl N-oxide, —C≡N, —CX′₃, —C(O)X′, —COOH, —COOR′, —C(O)R′, —C(O)NH₂,
—C(O)NHR′, —C(O)NR′₂, —C(O)H, —P(O)(OR′)OR″ and X′;
wherein X′ is selected from the group consisting of halogen and R, R′ and R″ are each independently selected from the group consisting of hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

19. The method of claim 17, wherein the compound is dimethylfumarate.

20-23. (canceled)

24. The method of claim 17, wherein the subject is monitored for an increase in level of frataxin responsive to the administering.

25-53. (canceled)
